(19) United States
(12) Patent Application Publication
(10) Pub. No.: US 2026/0033828 A1
(43) Pub. Date: Feb. 5, 2026

(54) DEVICE FOR JOINING FLAPS OF A HEART VALVE

(71) Applicant: Star Tric S.r.l., Milano (MI) (IT)

(72) Inventors: Marco GARD, Borgomasino (TO) (IT); Matteo ANTONIOTTI, Settimo Torinese (TO) (IT); Daniele ZANOTTI, Comelico Superiore (BL) (IT); Michele DE BONIS, Cernusco sul Naviglio (MI) (IT); Ottavio ALFIERI, Brescia (BS) (IT); Eugenio Maria PASSANANTE, Milano (MI) (IT)

(73) Assignee: Star Tric S.r.l., Milano (MI) (IT)

(21) Appl. No.: 19/151,545

(22) PCT Filed: Nov. 7, 2023
(86) PCT No.: PCT/IB2023/061207

§ 371 Date: Jul. 28, 2025

(30) Foreign Application Priority Data
Nov. 8, 2022 (IT) 102022000023010

(51) Int. Cl. A61B 17/04; A61B 17/00; A61M 25/00; A61M 25/06; A61M 29/02

(52) U.S. Cl. CPC A61B 17/0469; A61B 17/0482; A61B 2017/00243; A61B 2017/00292; A61B 2017/00349; A61B 2017/00867; A61M 25/0074; A61M 2025/0095; A61M 2025/0681; A61M 29/02

(57) ABSTRACT

A device for joining the leaflets of a heart valve together by joining them together while they are held aligned along the valve plane is equipped with a plurality of arms. Each arm is configured to assume a radially expanded position so that, when they are in the radially expanded position, they define as many radially oriented coplanar supports to support valve flaps.

### TECHNICAL FIELD

This disclosure relates to the repair of heart valves showing regurgitation. More particularly, the invention relates to an apparatus suitable for a less invasive repair of a heart valve using a device,, that may be positioned through a catheter, for catching the flaps of a heart valve, for example a tricuspid or mitral valve.

### BACKGROUND

The most common type of tricuspid valve dysfunction is functional tricuspid regurgitation (TR), which is mainly due to dilation of the tricuspid annulus after the dilation of the right ventricle. Later in the course of the disease, the “tethering” of the tricuspid flaps may also take place due to the dislocation of the papillary muscles within the remodeled right ventricle. When the functional TR is due to both severe annular dilation and flap chaining, annuloplasty alone is unlikely to be effective. Similarly, TR caused by prolapse or “flail” of multiple flaps, as typically seen in post-traumatic TR and severe degenerative TR, cannot be corrected by a simple annuloplasty procedure. To obtain an effective and lasting repair, the so-called “clover” technique has been proposed. This technique consists of tying together the central part of the free edges of the tricuspid flaps, producing a valve in the shape of a “clover”. A visual representation of a tricuspid valve treated according to this technique is shown in FIG. 1. Clinical results obtained with this technique are reported in:
“The ‘clover technique’ as a novel approach for correction of post-traumatic tricuspid regurgitation”, O. Alfieri, M. De Bonis et al., Journal of Thoracic and Cardiovascular Surgery, 2003; Vol. 126, No. 1, pages 75-79;
“A novel technique for correction of severe tricuspid valve regurgitation due to complex lesions.” De Bonis M, Lapenna E et al. Eur. J. Cardiothorac. Surg. 2004 May;25(5):760-5.
“Four-leaflet clover repair of severe tricuspid valve regurgitation due to complex lesions”, E. Lapenna, M. De Bonis et al., Journal of Cardiovascular Medicine, 2008, Vo. 9 No. 8, pages 847-849;
“The clover technique for the treatment of complex tricuspid valve insufficiency: midterm clinical and echocardiographic results in 66 patients”, E. Lapenna, M. De Bonis et al., European Journal of Cardio-thoracic Surgery, 37 (2010), 1297-1303;
“Long-term results (up to 14 years) of the clover technique for the treatment of complex tricuspid valve regurgitation”, De Bonis M, Lapenna E, et al. Eur. J. Cardiothorac. Surg. 2017 Feb. 23. doi: 10.1093/ejcts/ezx027.

Devices for catching opposite flaps of a mitral valve as well as a tricuspid valve are sold under the trade names MITRACLIP™ and TRICLIP™. This known device, which can be introduced into the heart through a catheter with a vascular approach or through a small incision in the chest, comprises an applicator of a catching device of the type shown in FIG. 2. The sequence of operations to be performed to implant a device MITRACLIP™ catching device is shown in FIG. 3. In the illustrated case the heart valve is the mitral valve, but the same observations apply mutatis mutandis also to the tricuspid valve. Using a catheter, the MITRACLIP™ catching device is inserted in a bent configuration into the heart; when the catheter is close to the heart valve, the latch is deployed like an umbrella to capture the valve flaps, and is subsequently closed to hold the flaps together. Finally, the MITRACLIP™ catching device is left closed in the heart to hold the flaps together, thereby reducing valve regurgitation.

Unfortunately, tests performed by the Applicant have shown that this known applicator is not capable of simultaneously catching all three flaps of the tricuspid valve, therefore, its effectiveness in treating tricuspid regurgitation is very limited. In the presence of a highly dilated tricuspid annulus, catching only two tricuspid valve flaps is also difficult with the MITRACLIP™ system.

Instead, it would be desirable to have a device that allows the leaflets of the tricuspid (or mitral) valve to be connected to each other exactly as shown in FIG. 9, keeping the leaflets coplanar.

A device for joining together the leaflets of a tricuspid valve, or even a mitral valve, is disclosed in the International patent application PCT/IB2022/054572 in the name of the same Applicant.

A limitation of this known device is that it does not allow surgeons to easily establish the direction along which to point the internal catheters 10 to establish at which point to harpoon heart valve leaflets. In theory, the point at which the flaps are harpooned could be established by inserting the device 1 more or less deeply beyond the valve plane, but such targeting is not simple.

### SUMMARY

An objective of this disclosure is to provide an alternative device for joining the leaflets of a heart valve by connecting them together while held aligned along the valve plane. This exceptional result is achieved with a device as defined in the attached claim 1. Further embodiments are defined in the attached claims.

### BRIEF DESCRIPTION OF THE DRAWINGS

FIG. 1 schematically shows a typical configuration of a tricuspid valve after surgery with the so-called “clover” technique.

FIG. 2 shows a known attachment device for flaps of a tricuspid or mitral valve.

FIG. 3 shows various steps for implanting the so-called MITRACLIP™ catching device to the flaps of a heart valve.

FIGS. 4a and 4b are views taken from different points of a device of this disclosure in closed configuration, with unbent arms.

FIGS. 5a and 5b are views taken from different points of a device of this disclosure in an unbent configuration, with the arms in an unbent configuration to define a resting plane for heart valve flaps.

FIGS. 6a and 6b are views taken from different points of a device of this disclosure in an unbent configuration and with catheters emerging from the cylindrical proximal body of the device.

FIG. 6c is a sectional view of the device of FIGS. 6a and 6b showing a catheter that emerges from the cylindrical proximal body.

FIGS. 7a and 7b show the device of FIGS. 4a and 4b inserted between the flaps of a tricuspid valve.

FIGS. 8a and 8b show the device of FIGS. 5a and 5b inserted between the flaps of a tricuspid valve, with the arms bent to form an elbow which define a support plane for the flaps of the tricuspid valve.

FIGS. 9a and 9b show the device of FIGS. 6a and 6b inserted between the flaps of a tricuspid valve, with the catheters crossing the flaps of the tricuspid valve.

FIG. 10 shows a device of this disclosure in an unfolded configuration and with a translatable solid body, with the arms bent at an elbow to define a support surface for heart valve leaflets.

FIG. 11 is a view from the rear of the cylindrical proximal body of a device of this disclosure showing the coaxial rod, the first coaxial tube, the second coaxial tube, an internal catheter inserted into through channels of the cylindrical proximal body.

FIG. 12 shows the device of FIG. 10 with a translatable solid body partially retracted to bend the internal catheter to make it diverge from the central longitudinal axis of the device.

FIG. 13 is an axial longitudinal section view of the device of FIG. 12, showing how the frusto-conical body causes the internal catheter to diverge as it slides along its inclined lateral surface.

FIGS. 14a and 14b show the device of FIG. 12 with an internal catheter bent to diverge from a central longitudinal axis and with an anchor element outside the catheter in a radially expanded configuration.

FIGS. 15a and 15b illustrate another embodiment in which the intermediate body 24 is fixed to the proximal body 3 and is kept at a pre-established distance from it through a plurality of bars 33 arranged longitudinally.

FIGS. 16a and 16b refer to an embodiment in which an inflatable member is inflated to cause an internal catheter to diverge from a central longitudinal axis.

FIG. 16c shows the inflatable balloon of FIGS. 16a and 16b when deflated.

FIGS. 17a to 17f show a frame comprising a series of self-expanding fins which can pass from a radially contracted to a radially expanded condition and respective rings adapted to guide the internal catheters to slide along the fins causing them to diverge from a central longitudinal axis.

FIG. 18a shows an anchoring element within a load-bearing catheter (in semi- transparency) in a longitudinally extended configuration.

FIG. 18b shows an anchoring element on the outside of a load-bearing catheter in a radially expanded configuration.

FIGS. 19a and 20a show anchoring elements in a longitudinally extended configuration obtained through longitudinal cuts on a side wall of an elastic or shape memory tubular element.

FIGS. 19b and 20b show anchoring elements in a radially expanded configuration.

FIG. 21a is a proximal view of the anchoring element of FIG. 19b connected to a thread-like connection element.

FIG. 21b is a sectional view of the anchoring element of FIG. 21a which shows the thread-like connection element embedded in a block of solid resin and this block of solid resin is interlocked to the internal walls of the anchoring element by means of a solid central strut.

FIGS. 22a to 22d show anchoring elements made of inflatable balloons.

FIG. 23 shows some of the shapes that balloon anchors can take once inflated.

FIGS. 24a and 24b show a possible construction method of the inflation tube of the balloon fasteners, such that the point at which the tube can be separated from the balloon once inflated is predefined.

FIGS. 25a to 25d show hinged arms with an elbow joint which may be bent by rotating around the joint to define a resting plane of the flaps of a heart valve, usable in the device of this disclosure.

FIGS. 26a to 26f show hinged arms with an elbow joint and a free protruding end which may be bent by rotating around the joint until they assume a hook shape which may be used in the catching device of this disclosure so as to tighten the flaps of a heart valve against the cylindrical proximal body 3.

FIGS. 27a to 27f show flexible integral arms that may be elastically deformed until they assume a hook shape, that may be used in the device of this disclosure so as to tighten the flaps of a heart valve against the cylindrical proximal body 3.

FIGS. 28a, 28b and 28c show the thread-like connecting elements connected to the leaflets of a tricuspid valve positioned with the device of this disclosure.

FIGS. 29a through 29f show how to position a holding device operable with the device of this disclosure to tension the thread-like connecting members at the center of a tricuspid valve.

FIG. 29g is a top view of a holding device flare washer of FIGS. 29a through 29f positioned in the center of a tricuspid valve.

FIGS. 30a to 30g show how to hold and cut the thread-like connecting elements using the cutting and holding device of FIGS. 29a to 29g.

FIGS. 31a to 31c show how the thread-like connection elements are held and cut thanks to the cooperation between a screw and a countersunk washer with a central through hole with a nut thread of the cutting and holding device of FIGS. 29a to 29g.

FIGS. 32a to 32d show a section of a cutting and holding device for cutting and holding the threads, usable with the device of FIG. 6a, based on the cooperation between a screw and a countersunk washer having a central through hole with a thread nut and a device blade that can rotate circumferentially.

FIGS. 33a to 33d show a detail of another cutting and holding device similar to that of FIGS. 32a to 32d, in which the screw stem is also internally threaded, with a threaded pin screwed into the screw and in the washer.

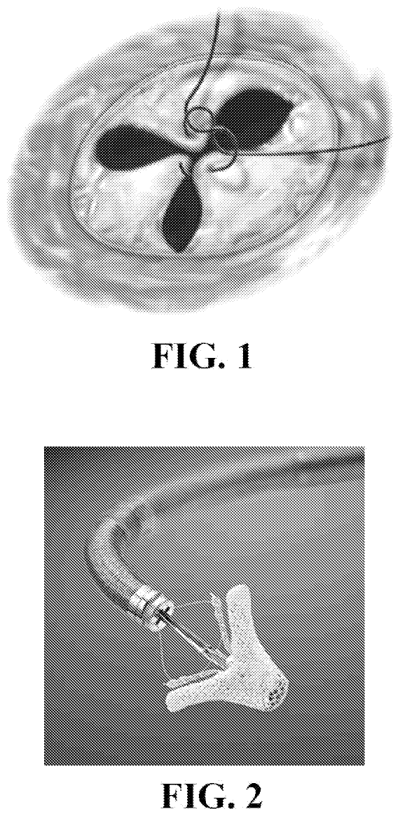

### DESCRIPTION OF ILLUSTRATIVE EMBODIMENTS OF THE INVENTION

An embodiment of a device for joining together the flaps of a tricuspid valve, or even of a mitral valve, will be illustrated with reference to the enclosed figures.

It comprises an external catheter 2 suitable for being inserted into a patient's blood vessel, that performs the function of a container which carries inside the blood vessel the internal part for joining the flaps together. The internal part comprises a cylindrical proximal body 3, which defines a central axial cavity 4 which runs in the cylindrical proximal body 3 from a first base surface to a second base surface, as well as a cylindrical distal body 5, which has a rigid coaxial shaft 6 which is fixed and rises from a central position of a base surface of the cylindrical distal body 5 and which is slidably inserted into the central axial cavity 4 of the cylindrical proximal body 3. In practice, by pushing or pulling the rigid coaxial shaft 6 it is possible to move the cylindrical distal body 5 away or closer to the cylindrical proximal body 3. The device further has a deflector body 26 which defines a second central axial through hole running therethrough and has a side surface configured so that, when the diverter body 26 is outside the external catheter 2, pushing an internal catheter 10 in the longitudinal direction, the internal catheter 10 is forced to bend as a result of the contact with the lateral surface of the guide to slide along the deflector body 26, being guided by the contact with the lateral surface itself bending so as to diverge from the longitudinal axis central part of the device 1. There is also an intermediate body 24, defining a third central axial through opening which runs from a first base surface to a second base surface. A second coaxial tube 25 is fixed and rises from a central position of the first base surface of the intermediate body 24 and is in communication with the third axial through hole.

The proximal cylindrical body 3 has a portion integral but spatially separated longitudinally in the distal direction, called intermediate cylindrical body 24.

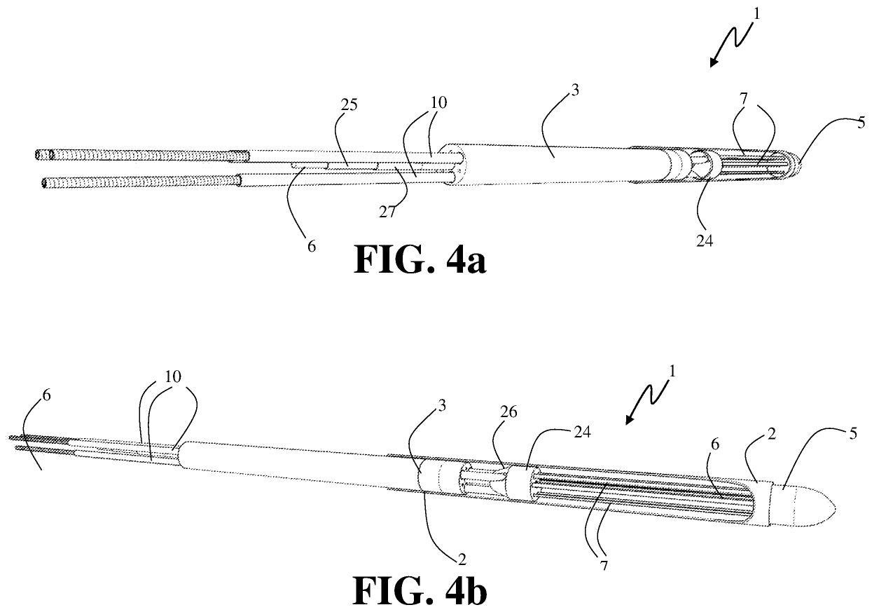

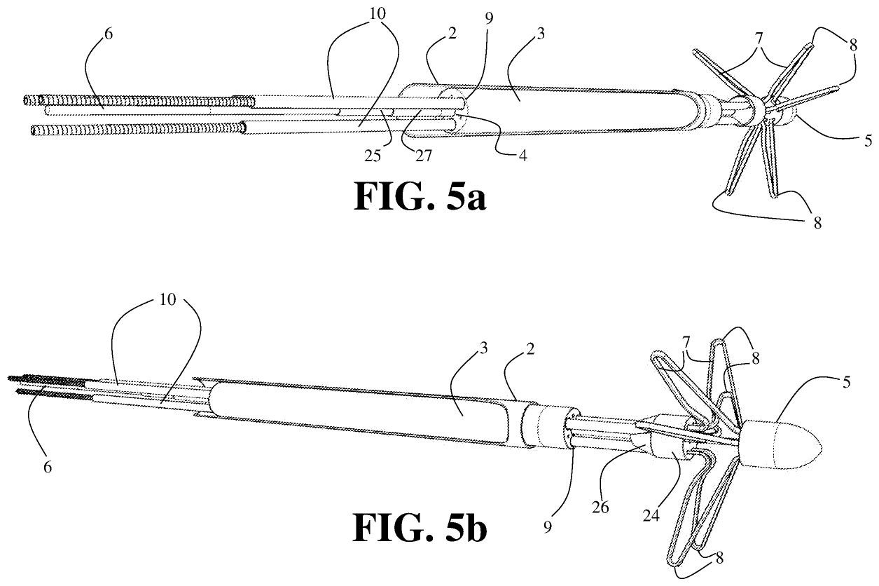

Between the two cylindrical bodies 5 and 24, several flexible arms 7 are installed which have a first end fixed on a base surface of the cylindrical intermediate body 24, and a second end fixed to the cylindrical distal body 5. Each arm is configured to spontaneously assume a radially expanded position with an elbow 8 when exiting the external catheter 2, as shown in FIGS. 5a and 5b, and to assume an extended position parallel to the rigid coaxial shaft 6 when both the cylindrical intermediate body 3 and the cylindrical distal body 5 are inside the external catheter 2, as shown in FIGS. 4a and 4b.

The arms 7 are configured in such a way as to define, when they are in the radially expanded position, coplanar supports radially oriented to support the flaps of a tricuspid or mitral valve, keeping them substantially aligned with the plane identified by the supports.

In the cylindrical proximal body 3 there are at least two through channels 9 which run in the cylindrical proximal body 3 from as many inlet holes in the first base surface of the cylindrical proximal body 3 to as many outlet holes.

Each of these through channels 9 is connected to a respective internal catheter 10, so as to be able to convey a thread-like connection element through the internal catheter 10 and to direct it at least partially in the radial direction making it come out of the respective outlet hole from the lateral surface of the cylindrical proximal body 3.

For example, a thread-like connection element 11 can be a suture thread, or a flexible rod, or even a tubular element, etc.

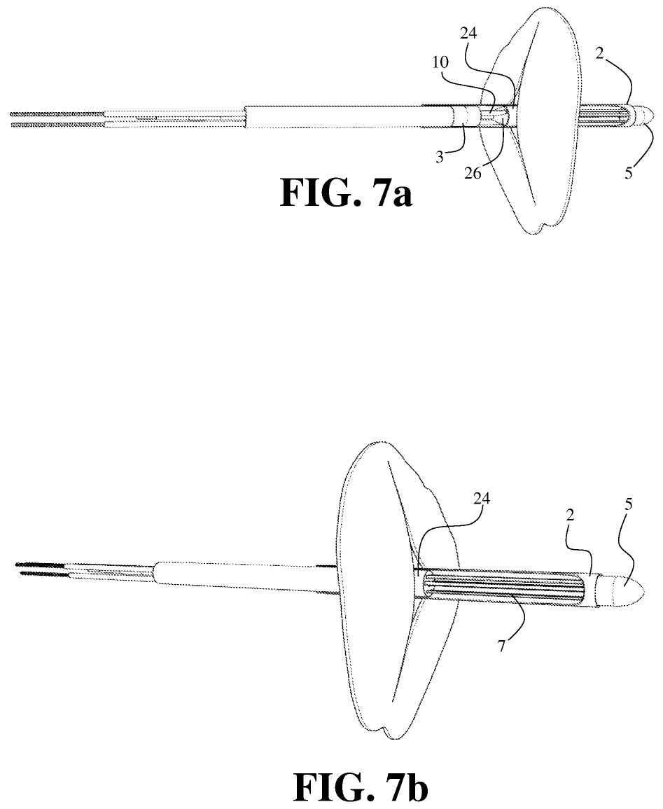

To better understand how the device of FIGS. 4a to 6c of this disclosure is used to join together the flaps of a heart valve, reference will be made to FIGS. 7a to 9c which show the use of the device 1 to join the three flaps of a tricuspid valve.

The closed device 1 is pushed through a blood vessel until it reaches the heart valve whose flaps must be tied together, positioning it so that the external catheter 2 crosses the valve plane and the opening of the external catheter 2 is beyond this plane, so as to open below the flaps to be tied together, as shown in FIGS. 7a and 7b. Keeping the external catheter 2 in this position, the rigid coaxial shaft 6 is pushed so as to make the cylindrical distal body 5 come out of the external catheter 2.

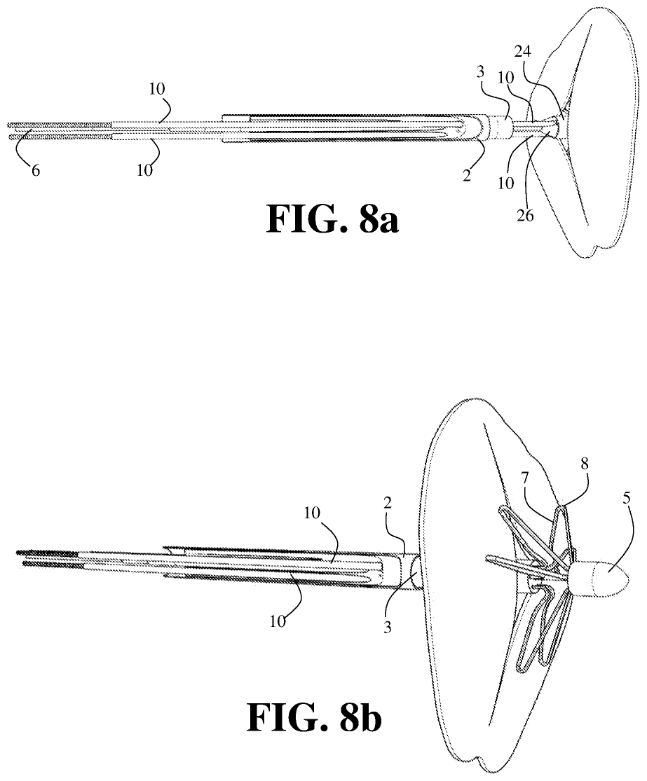

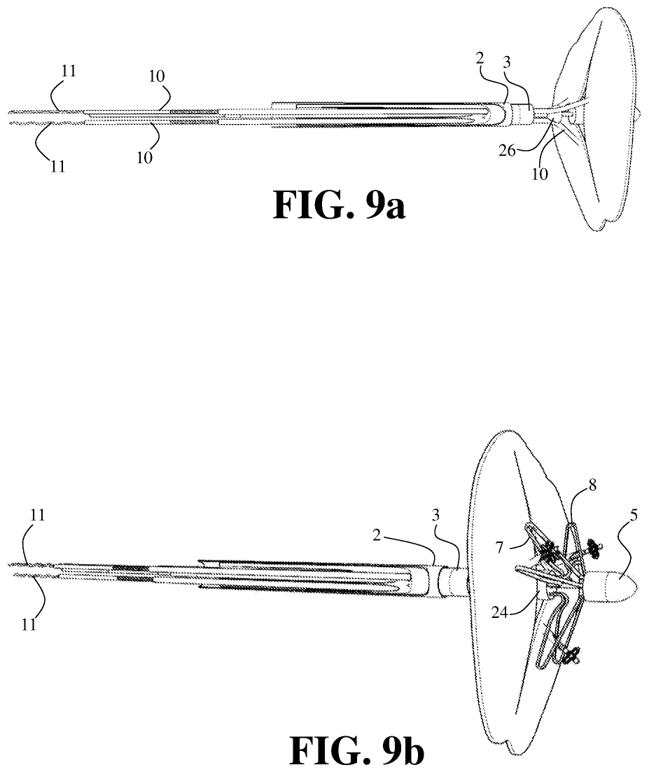

Since the arms 7 outside the external catheter 2 are free to expand, they assume a radially expanded position with an elbow bend 8, define radially oriented coplanar supports to sustain the flaps of a heart valve, for example tricuspid or mitral valve. As shown in FIGS. 8a and 8b, the flaps of the tricuspid valve rest on the bent arms 7. By pulling the rigid coaxial shaft 6, a surgeon may raise the bent arms 7 so as to bring the flaps back into the valve plane. The device 1 of this disclosure is therefore structured to support the valve flaps without interfering with the chordae tendineae of the valve itself (not shown in the figures) which are located on the opposite side with respect to the bent arms 7. When the flaps are aligned with the plane of the valve, internal catheters 10 having inside thread-like connection elements 11, are made to come out of the holes in the lateral surface of the cylindrical proximal body 3 so as to capture the flaps, as shown in FIGS. 9a and 9b.

The deflector body 26, along which the internal catheters 10 slide when pushed longitudinally, can be made in different ways in order to deflect the catheters 10 making them diverge from the central longitudinal axis of the device 1. For example, it can be a translatable solid, or it can be an inflatable/deflatable element, or even a frame equipped with self-expanding fins and guide rings for the internal catheters.

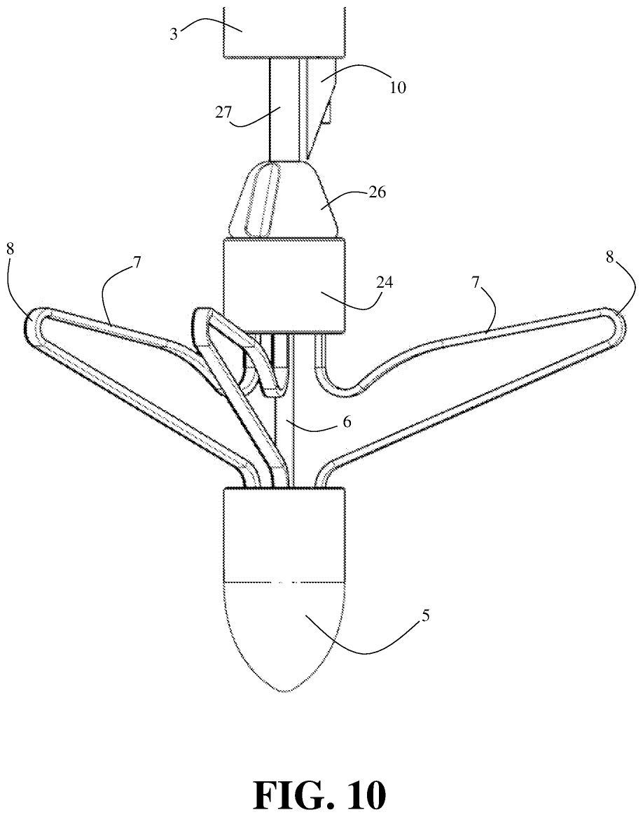

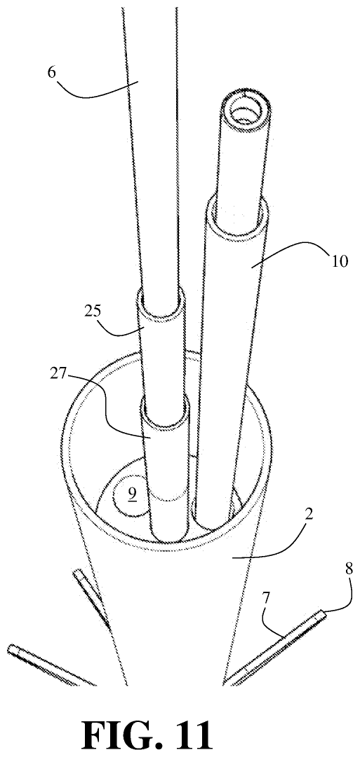

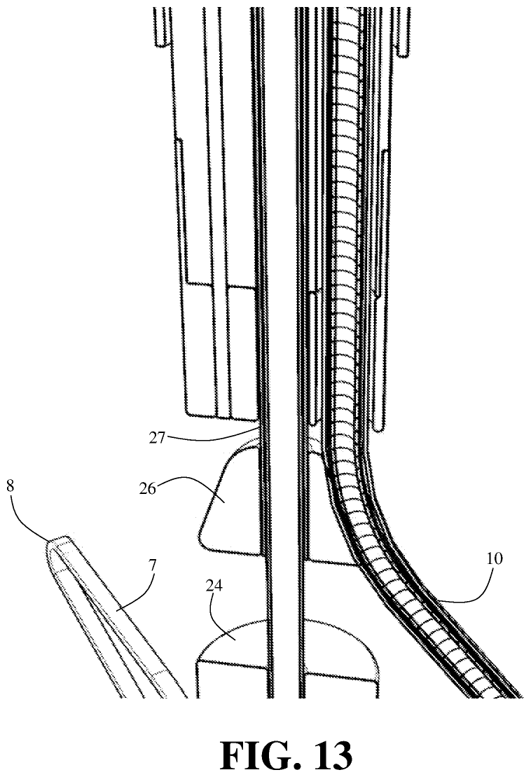

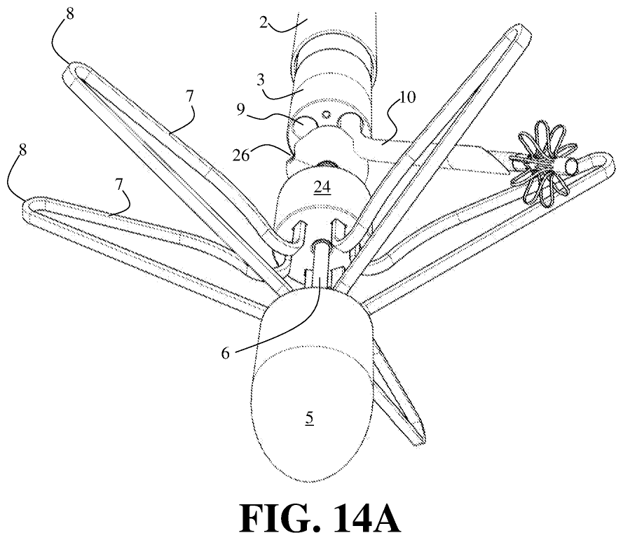

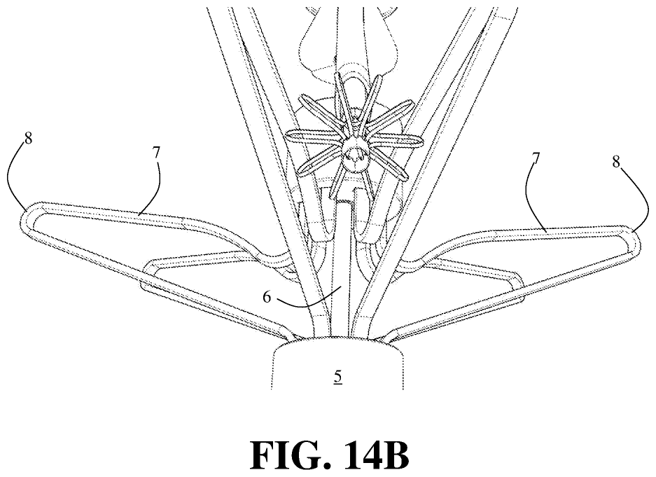

According to one aspect, illustrated in particular in FIGS. 10 to 14b, the deflector body 26 is a translatable solid 26, for example substantially frusto-conical in shape, having a smaller transverse base surface, a larger transversal base surface and a lateral between the smaller transversal base surface and the larger transversal base surface. The translatable solid 26 is configured so that the internal catheters 10 are guided to slide along its lateral surface so as to progressively diverge in the transverse direction as they are pushed forward in the longitudinal direction of the device.

The translatable solid 26 has a first coaxial tube 27, which is fixed and rises from a central position of the distal surface of the solid 26 and is in communication with the respective axial cavity. The coaxial tube 27 is slidably inserted into the central axial cavity of the cylindrical proximal body 3. Thanks to this configuration, the surgeon can independently move:
the distal body 5 through the rigid rod 6;
the translatable solid body 26 via the first coaxial tube 27.

By pushing the distal body 5, the intermediate body 24 and the distal portion of the proximal cylindrical body 3 outside the carrying catheter 2, the arms 7 are found without any external constriction element and can be brought into their radially expanded form, defining a support to keep the leaflets in the valve plane, exactly as in the known device of FIGS. 4a to 6c.

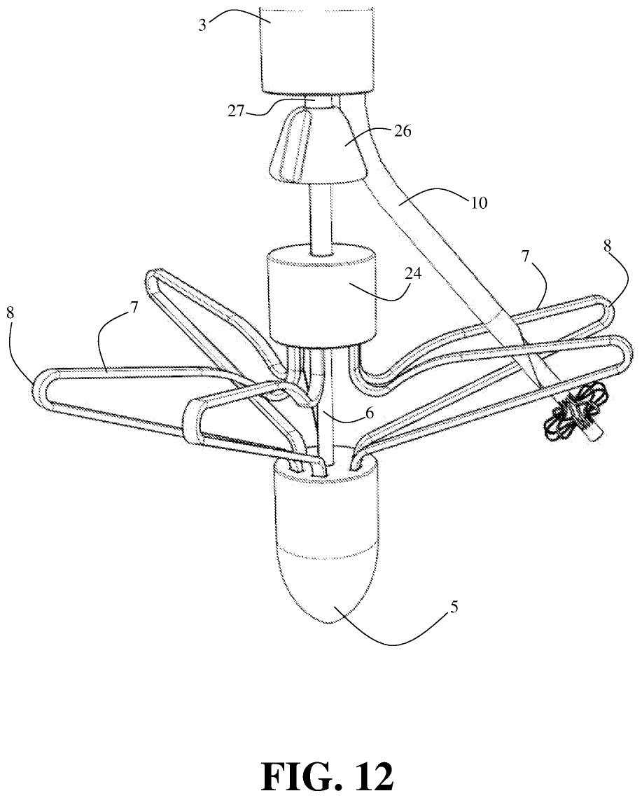

According to one aspect, the internal catheters 10 exit from the proximal body 3 parallel to the longitudinal axis of the device. However, thanks to the translatable solid 26, which can be moved axially by pulling or pushing the first coaxial tube 27, the internal catheters 10 are bent (FIG. 12) by sliding on the inclined lateral surface of the translatable solid 26. By pulling the translatable solid 26 axially, it is adjusted the distance with respect to the longitudinal axis at which the internal catheter 10 releases the hooking element 20 to hook the heart valve leaflet.

Optionally, to guide the sliding of each internal catheter 10 along the lateral surface of the translatable solid 26 and prevent it from moving circumferentially in an uncontrolled manner, an equal number of seats 28 can be defined on the translatable body 26, configured to accommodate the internal catheter 10 and maintain its orientation as it is pushed towards the arms 7.

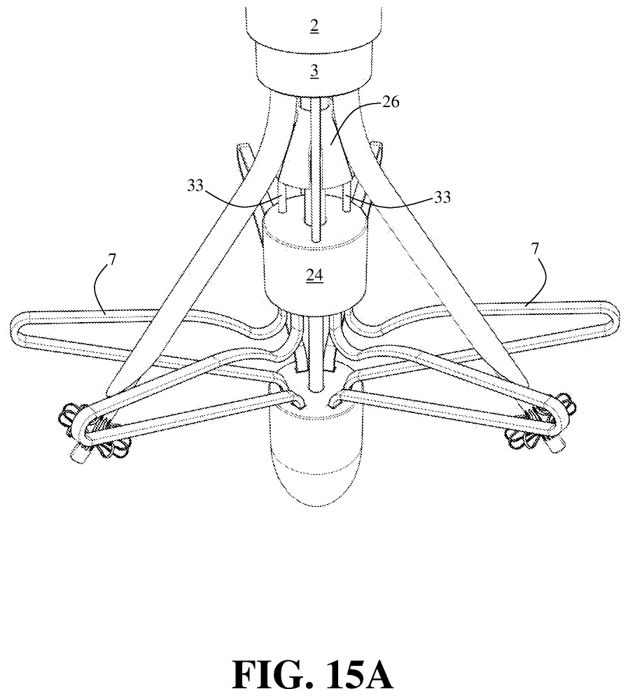

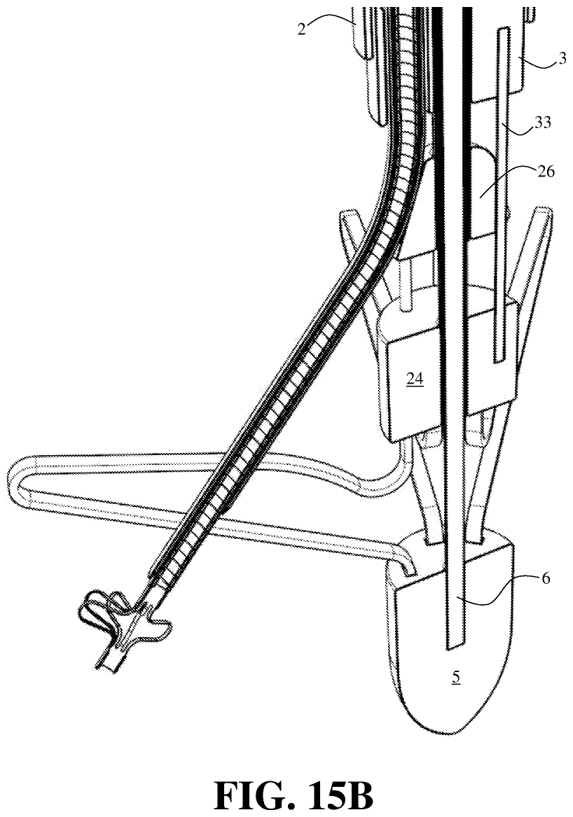

FIGS. 15a and 15b illustrate another embodiment in which the diverter body 26 is similar to that illustrated in FIGS. 10 to 14b and is configured so that the internal catheters 10 are guided to slide along its lateral surface in such a way to progressively diverge in the transverse direction as they are pushed forward in the longitudinal direction of the device. Differently from the embodiment of FIGS. 10-14b, the intermediate body 24 cannot be moved independently because it does not have the second coaxial tube 25, but is fixed to the proximal body 3 and is kept at a pre-established distance from it through a plurality of bars 33 arranged longitudinally and configured to engage the lateral surface of the deflector body 26 preventing it from rotating around the longitudinal direction. To this end, the bars 33 will be arranged so as to engage as many longitudinal seats, so the deflector body 26 will only be able to slide along the bars 33.

In this configuration the surgeon cannot independently move the proximal body 3 and the intermediate body 24, but will be able to retract or advance the intermediate body 24 by pulling back or pushing the proximal body 3 forward.

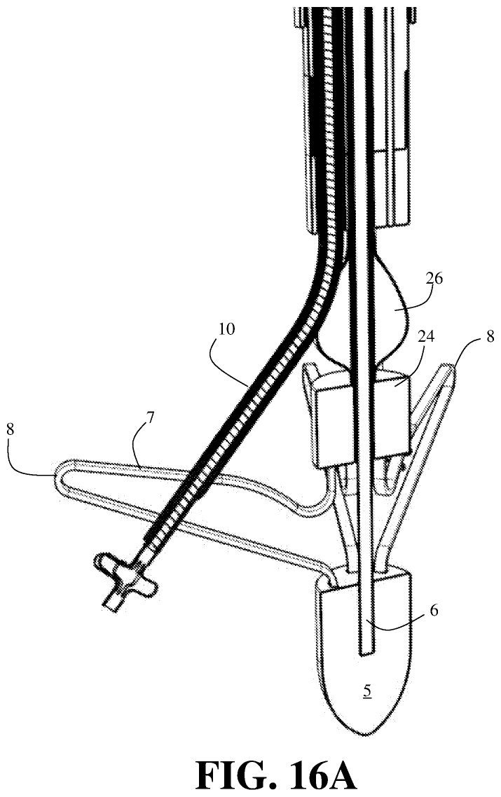

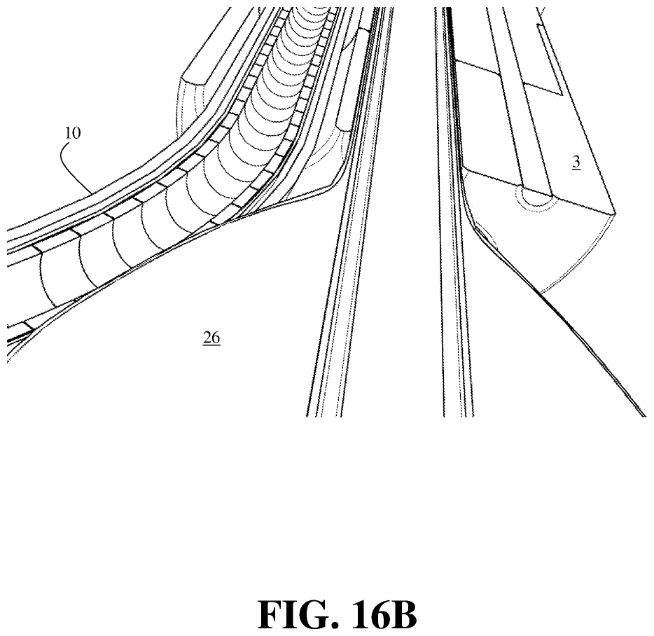

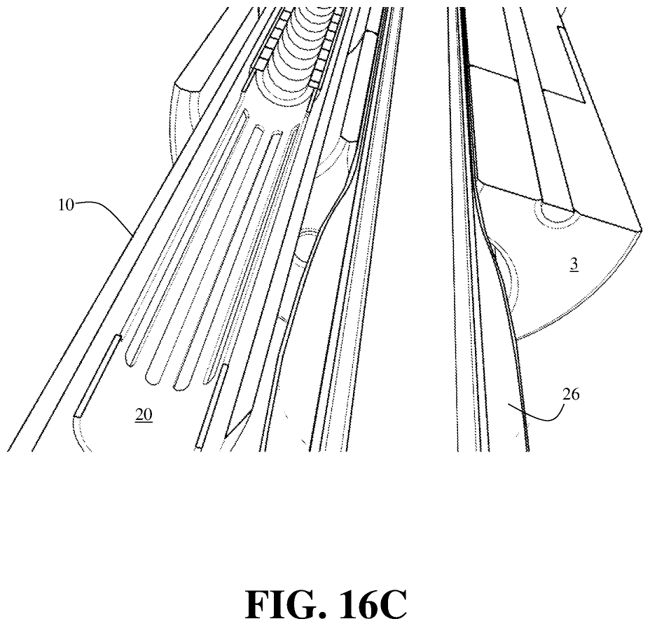

According to an alternative aspect illustrated in FIGS. 16a to 16c, the diverter body 26 is made by means of an inflatable/deflatable element 26. When the inflatable/deflatable element is inflated through the respective coaxial tube 27 (FIGS. 16a and 16b), it assumes an expanded shape surrounding the second coaxial tube 25 and defining a lateral surface with a smaller cross section at a proximal portion in front of the proximal body 3, and a larger cross section. Thanks to this expanded shape, the inflatable/deflatable element is configured to radially deflect and bend the internal catheters 10 outwards, as well as the translatable solid 26 of FIG. 12. Advantageously, the inflatable/deflatable element 26 can be deflated (FIG. 16c) using the coaxial tube 27, so as to be able to retract it inside the central axial cavity 4.

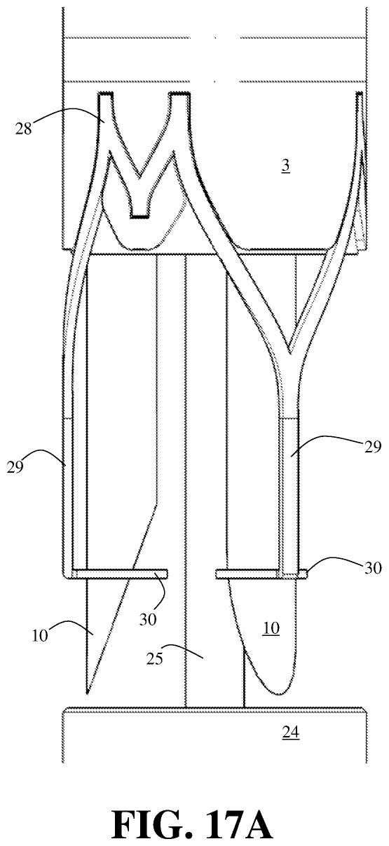

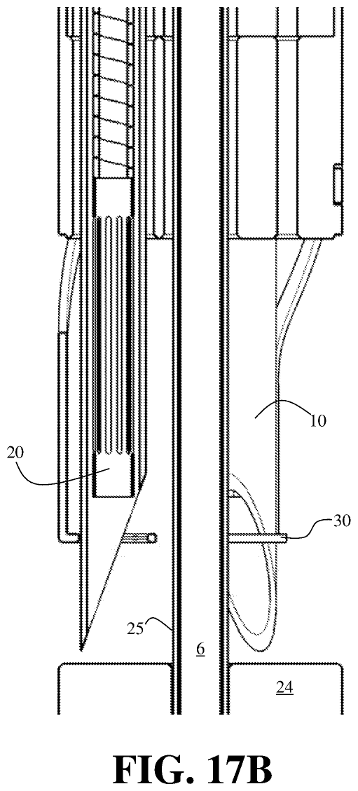

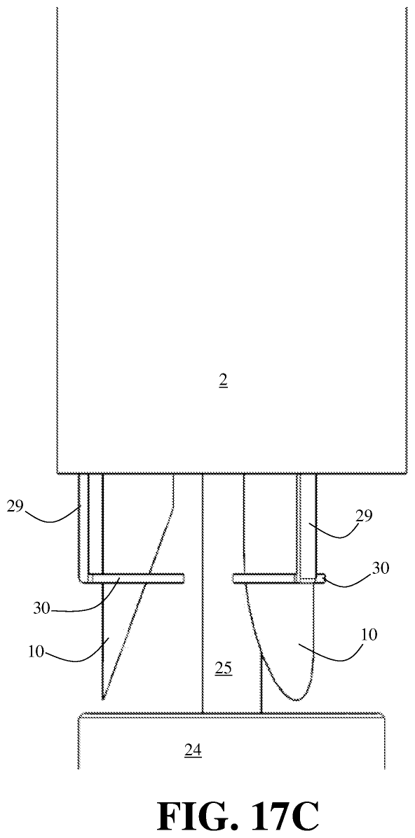

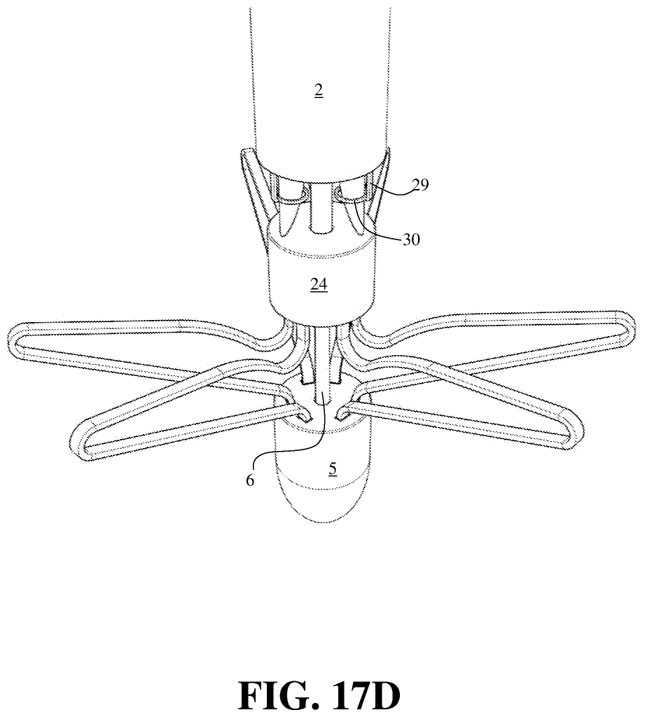

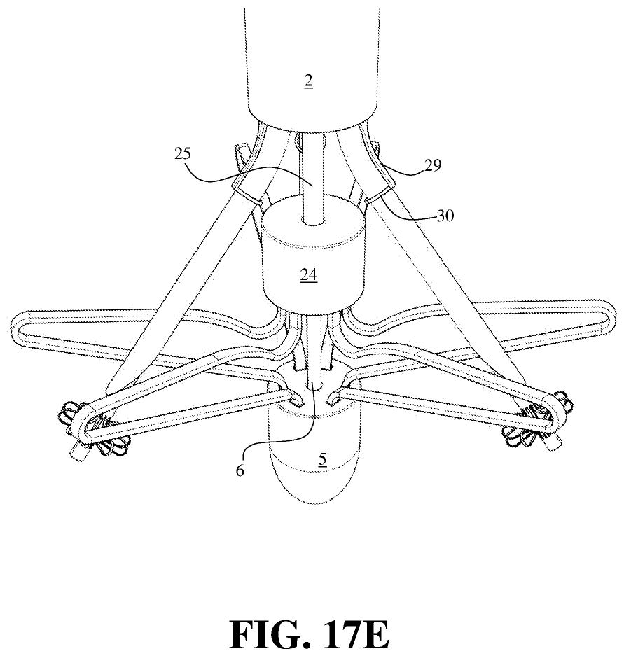

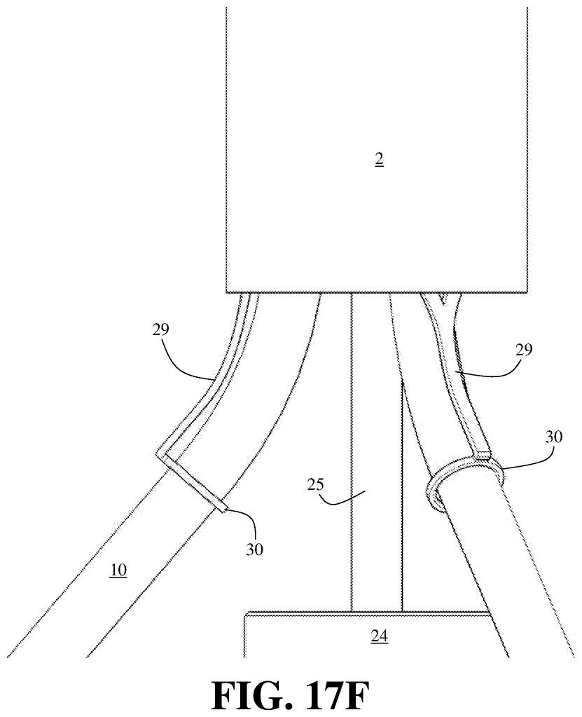

According to a further aspect illustrated in FIGS. 17a to 17f, the deflector body 26 includes a frame 28, fixed so as to transversely surround said proximal body 3, a plurality of fins 29 and as many rings 30, in which each fin 29 has a first end fixed to the frame 28 and a second end, opposite to the first end, integral with a respective ring 30. A respective internal catheter 10 is inserted into each ring 30 so that, when the internal catheter 10 is pushed in the longitudinal direction of the device, it is guided to slide along the fins 29. The coaxial tube 25 is fixed to the proximal body 3 so as to keep the intermediate body 24 and the proximal body 3 at a fixed distance and integral with each other.

The fins 29 are made so as to be self-expanding, for example they are made of shape memory material, and are configured so that, as the fins 29 protrude from the external catheter 2, the respective second ends always bend by more so as to diverge from the central longitudinal axis of the device, moving the rings 30 apart from each other, as illustrated in FIGS. 17e and 17f. Thanks to the fact that the fins 29 spontaneously diverge more and more in a radial pattern as they emerge from the external catheter 2, it allows the reciprocal distance between the rings 30 to be regulated and consequently the direction imparted to the internal catheters 10 to be regulated.

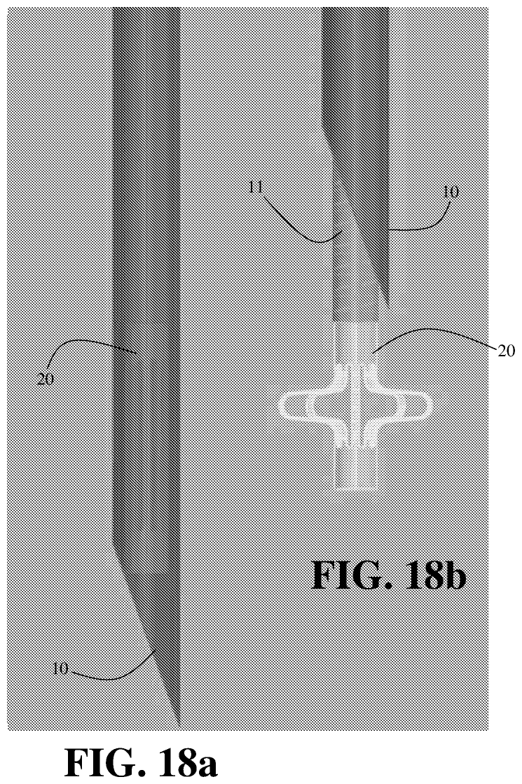

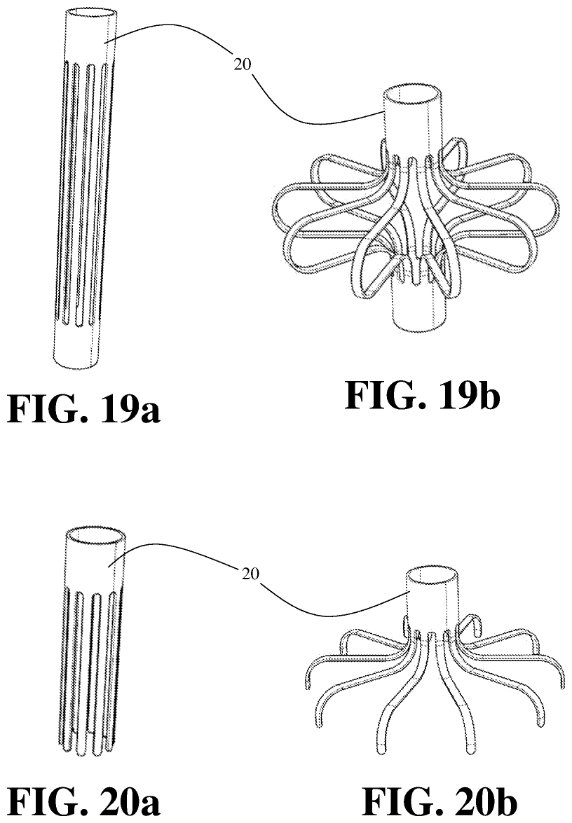

FIGS. 18a and 18b show an embodiment in which each filiform connection element ends with a coupling element 20, visible in FIG. 18a in a longitudinally extended semi- transparent configuration inside a carrying catheter 10 and in FIG. 18b at the exterior of a carrying catheter 10 in radially expanded configuration. According to one aspect, in the embodiment of FIGS. 18a and 18b the carrying catheter 10 is shaped like the tip of a syringe needle so as to pierce a heart valve leaflet entering from one face and release the coupling element 20 on the opposite face of the flap. The coupling element 20 is configured so as to remain longitudinally extended (FIG. 18a) when it is inside the carrying catheter 10, and to expand (FIG. 18b) radially when it is free. Once the hooking element 20 is free to expand, it can no longer pass through the hole made in the heart valve leaflet and therefore remains hooked to the leaflet and allows it to be pulled. FIGS. 19a and 20a show possible embodiments of such a coupling element 20, both obtained by cutting a tubular body made of elastic or shape memory material into longitudinal strips. In FIGS. 19a and 20a the longitudinal strips are in the longitudinally stretched configuration assumed when they are in the carrying catheter, and are configured to automatically expand radially when they are outside the internal carrying catheter 10. FIGS. 19b and 20b show the corresponding elements of coupling 20 of FIGS. 19a and 20a in radially expanded configuration. The shape that the coupling elements 20 can take in an expanded configuration can also be different from that shown. For example, the coupling element of FIG. 20b can assume a radially expanded configuration in which the strips form curls or are arranged to remain radially extended.

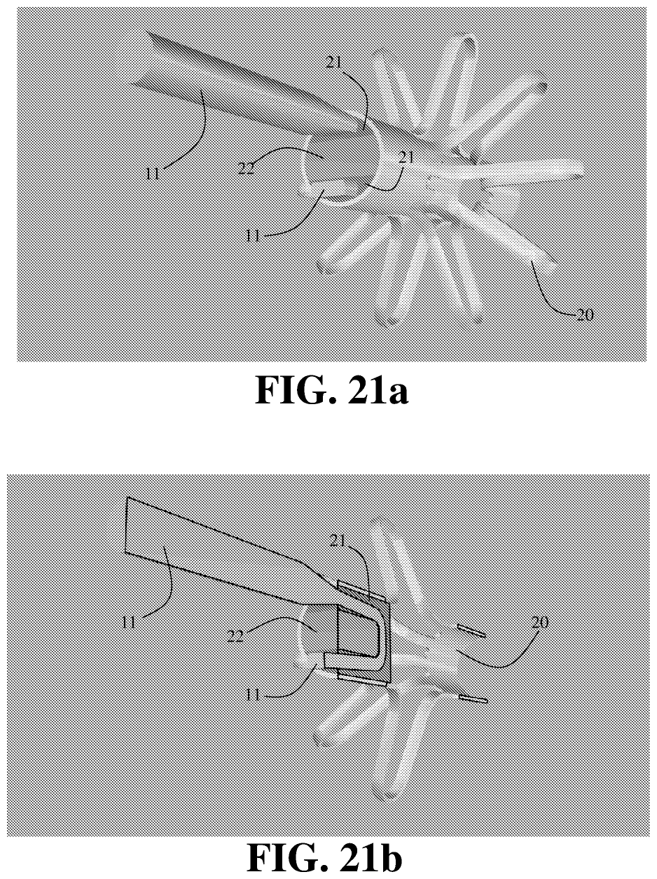

According to one aspect, each coupling element 20 of FIGS. 19a to 20b can be fixed to a respective thread-like connection element 11 in the way illustrated in FIGS. 21a and 21b. As illustrated in the sectional view of FIG. 21b, the thread-like connection element 11 is embedded in a block of solid resin 21. In turn, the block of solid resin 21 is fixed by interlocking to the internal walls of the anchoring element by a central body 22 wedged in the block of solid resin.

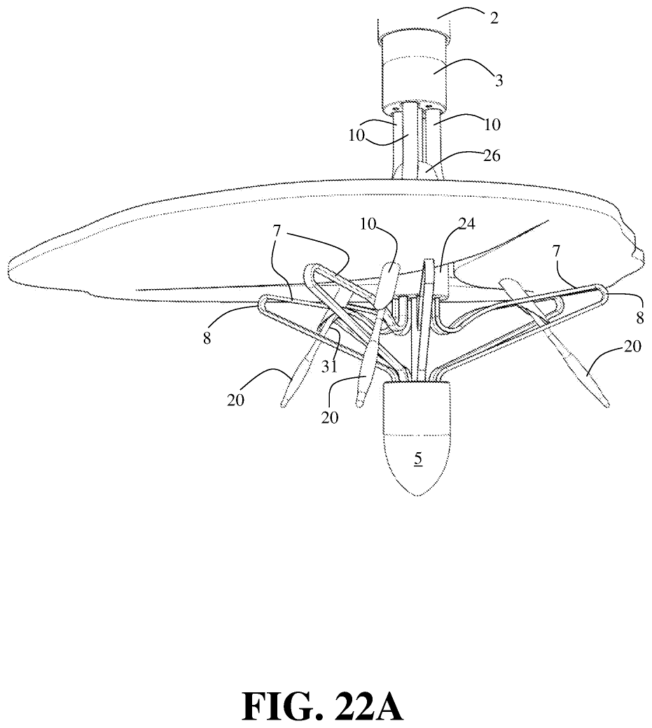

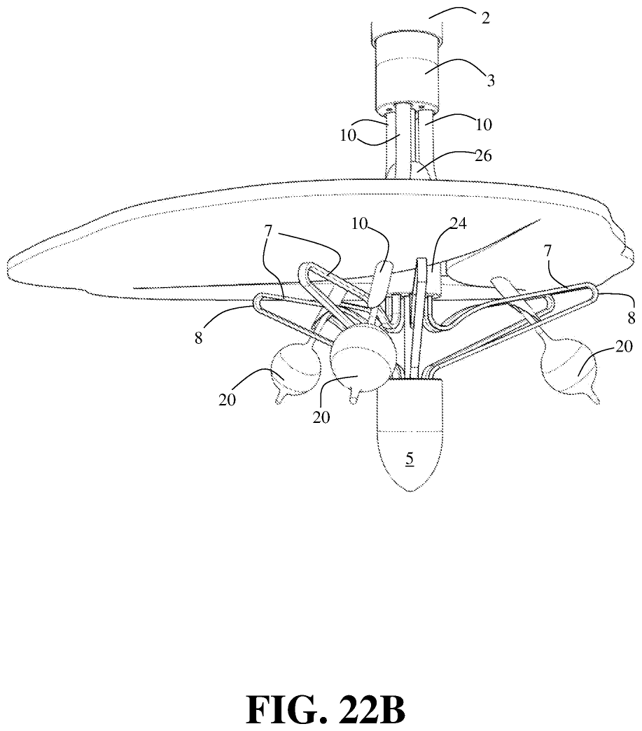

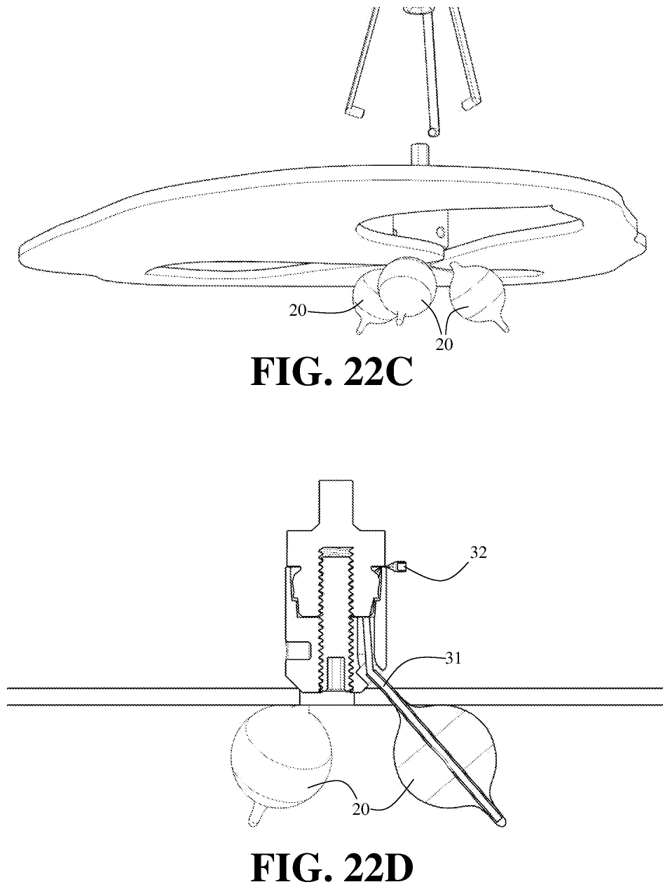

FIGS. 22a to 22d show a further embodiment in which the attachment elements 20 are made up of balloons, visible in their first contracted/deflated configuration (FIG. 22a) and then expanded/inflated (FIG. 22b). Each of the balloon attachment elements 20 is connected to a relative inflation tube 31 which, in this specific form of implementation, replaces the thread-like connection element 11 in the function of crossing and subsequently approaching and blocking the attached valve flaps. As in the case of the expandable elements 20, the balloons are also released by means of the carrying catheters 10 inside which they are inserted and can slide.

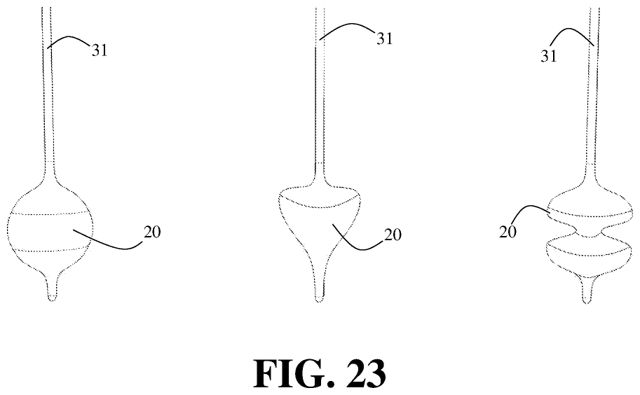

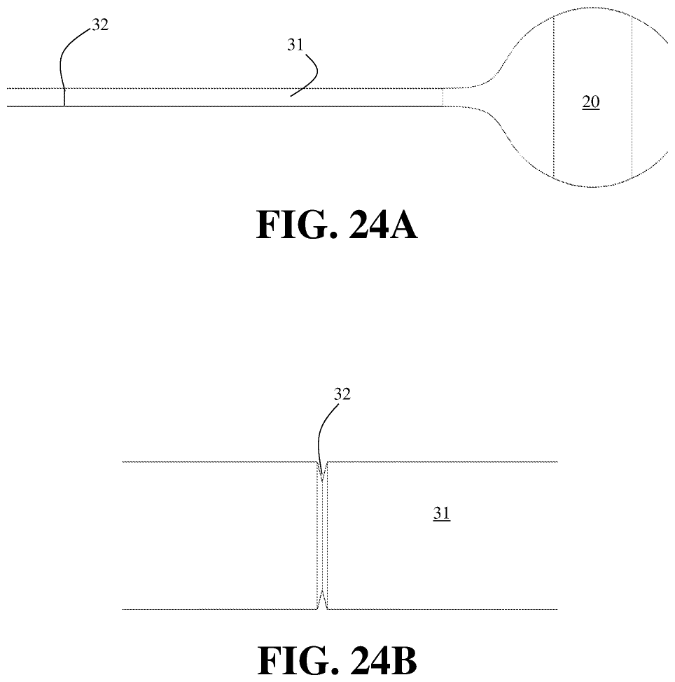

FIG. 23 shows possible embodiments of such a balloon attachment element, illustrating examples of shapes that these can take once inflated into the expanded form. FIGS. 24a and 24b illustrate a possible realization of the inflation tubes 31 of the balloons 20. Once each balloon coupling element 20 has been inflated and the respective inflation tube 31 has been choked and blocked by the holding device, it is necessary remove the proximal part of the inflation tube 31 which is not occupied by the holding device. To this end, according to an aspect shown in FIGS. 24a and 24b, the inflation tubes 31 are equipped with a pre-scored area 32 or an area in which the wall thickness of the inflation tube 31 is locally reduced. Thanks to this measure, when the inflation tube 31 is subjected to longitudinal traction beyond a certain pre-established force value, the tube 31 separates into two portions in correspondence with the pre-cut area 32 allowing the intact proximal portion of the inflation tube 31 to be extracted, while the distal portion of the inflation tube remains connected to the balloon coupling element 20 and remains engaged in the holding device (FIGS. 22c and 22d).

However, there may also be more than six arms 7, should a greater support of the tricuspid valve flaps be required, or there may be only two pairs of arms 7, for example to support the two flaps of a mitral valve. Similarly, there can also be only two internal through channels 9 in the cylindrical proximal body 3, to catch the two flaps of a mitral valve with as many suture threads that protrude from respective two holes in the lateral surface of the cylindrical proximal body 3.

According to one aspect, the arms 7 are made of a shape-memory material (for example, Nitinol). Optionally, they are composed of elastic material which gives an elbow-bent configuration at rest, as illustrated in FIGS. 5a and 5b, and can be configured in such a way as to be elastically forced to remain extended when they are inside the external catheter 2.

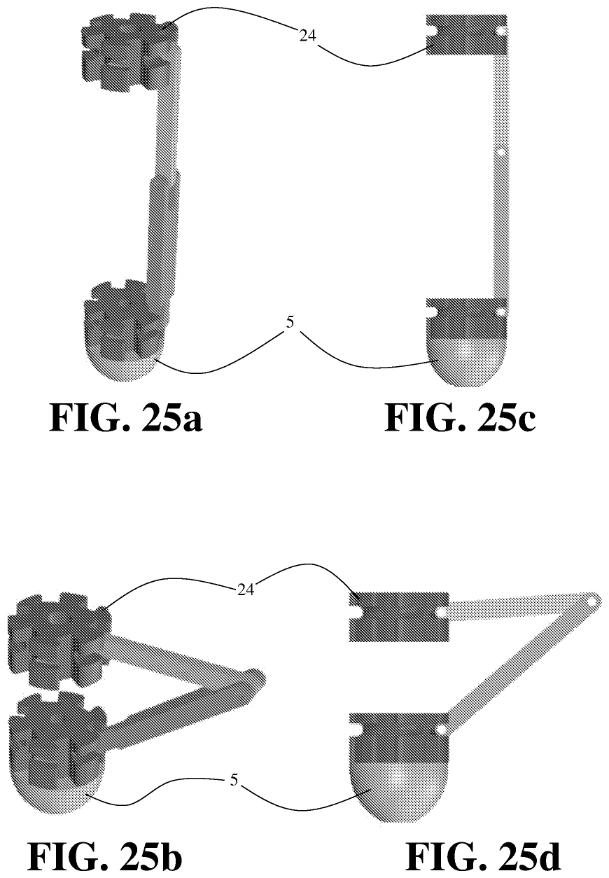

According to one aspect shown in FIGS. 25a to 25d, each arm is made by hinging a first rigid shaft at the first end of the arm on the second base surface of the cylindrical proximal body, and a second rigid shaft at the second end of the arm to the cylindrical distal body. The two shafts are hinged to each other at an intermediate area so that each arm can be bent forming the elbow 8 at the point where the two rods are hinged to each other. As in the case illustrated in FIGS. 9a and 9b, the bent arms define coplanar supports of the flaps of a heart valve.

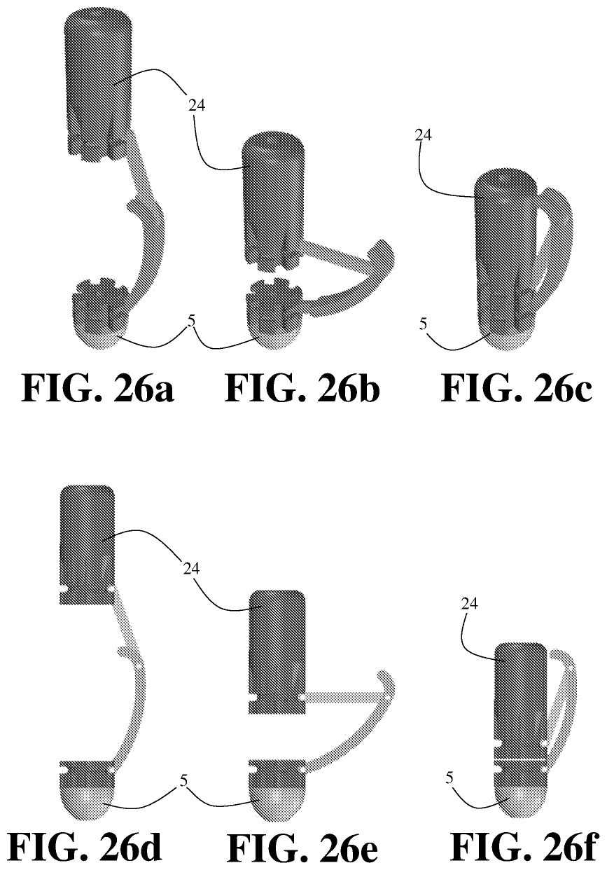

According to one aspect shown in FIGS. 26a to 26f, the arms are made with two rigid rods hinged to each other as in FIGS. 25a to 25d but, unlike the latter, the shaft connected to the cylindrical distal body 5 has a free end shaped as a hook. As indicated in the succession of FIGS. 26a to 26f, by progressively approaching the cylindrical proximal body 3 to the cylindrical intermediate body 24, the arm passes from the extended position (FIGS. 26a and 26d) to the radially expanded position (FIGS. 26b and 26e) forming an elbow 8, and further to a closed position (FIGS. 26c and 26f) in which the hook-shaped free end is functionally configured to cooperate with the coaxial shaft 6 to tighten a flap of a heart valve like two jaws. By making the arms as illustrated in FIGS. 26a to 26f, it is possible to keep the flaps in a plane (FIGS. 26b and 26e) or to pull them with the hook-shaped ends towards the coaxial shaft 6 by moving the distal body 5 towards the intermediate body 24 up to tighten the flaps between the coaxial shaft 6 and the hook-shaped end.

An advantage in making the arms in this way is the fact that, when the flaps are tightened between the coaxial shaft 6 and the respective hook-shaped end, they are well stretched and cannot move, making it easier to join them.

A further advantage is the fact that the flaps are pulled more towards the center of the valve plane and can be crossed by the respective suture wires in an area further away from the free end of the flap. Otherwise, the area in which the flaps are crossed by the respective thread-like connection elements will be determined by the inclination with which the internal catheters 10, which exit from the cylindrical proximal body 3, are deflected by sliding against the side surface of the frusto-conical body.

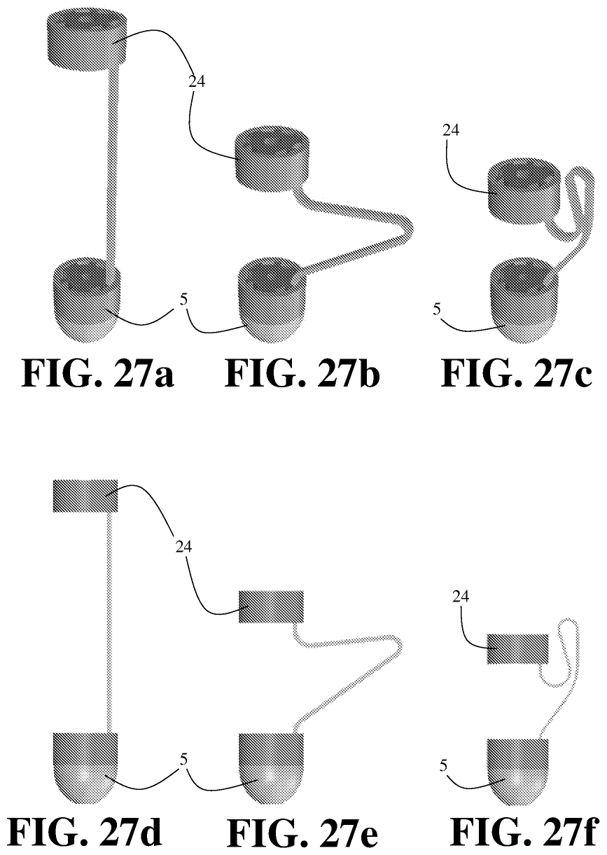

According to one aspect shown in FIGS. 27a to 27f, the arms are not constituted by two rigid hinged rods but are made in one piece with a shape memory material or with an elastically deformable material. As for the embodiment shown in FIGS. 26a to 26f, each arm is configured so that, by progressively approaching the cylindrical proximal body 3 to the cylindrical intermediate body 24, the arm passes from the extended position (FIGS. 27a and 27d) to the radially expanded position (FIGS. 27b and 27e) forming the elbow 8, and further to the closed position (FIGS. 27c and 27f) in which the elbow 8 is functionally configured to cooperate with the coaxial shaft 6 to tighten a flap like two jaws of a heart valve.

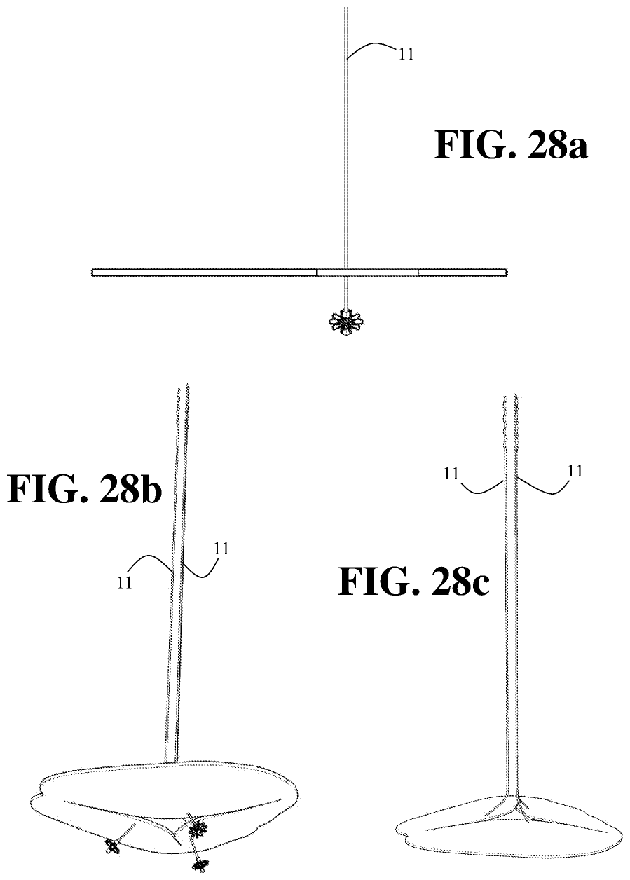

Once the flaps have been captured, the device 1 is removed leaving the thread like connection elements 11 as shown in FIGS. 28a and 28b. Subsequently, the thread-like elements are tied together and cut at a desired length, so that the valve flaps remain tied together near the center of the valve.

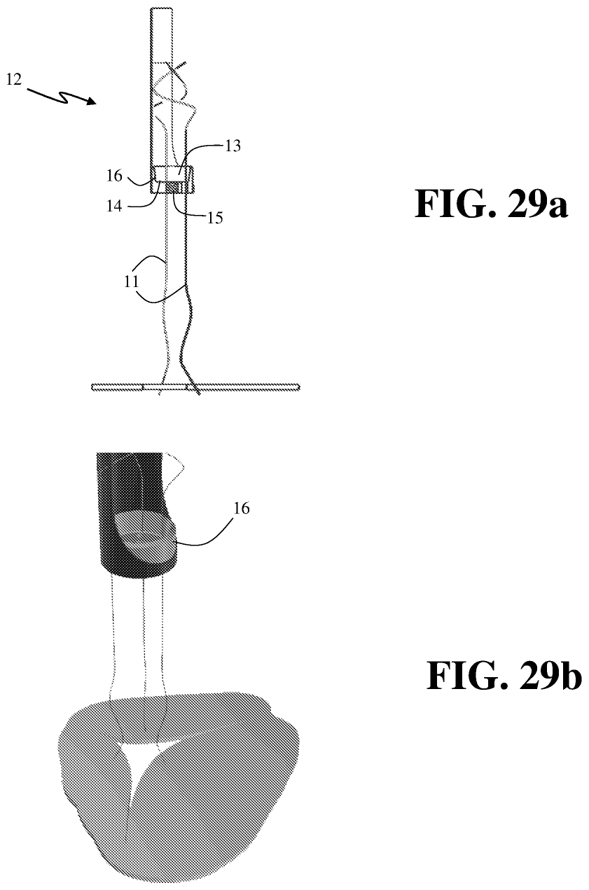

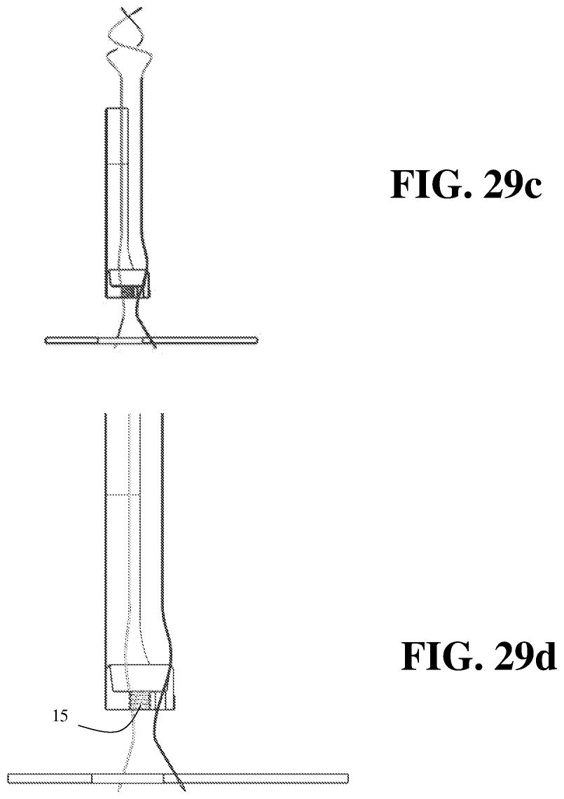

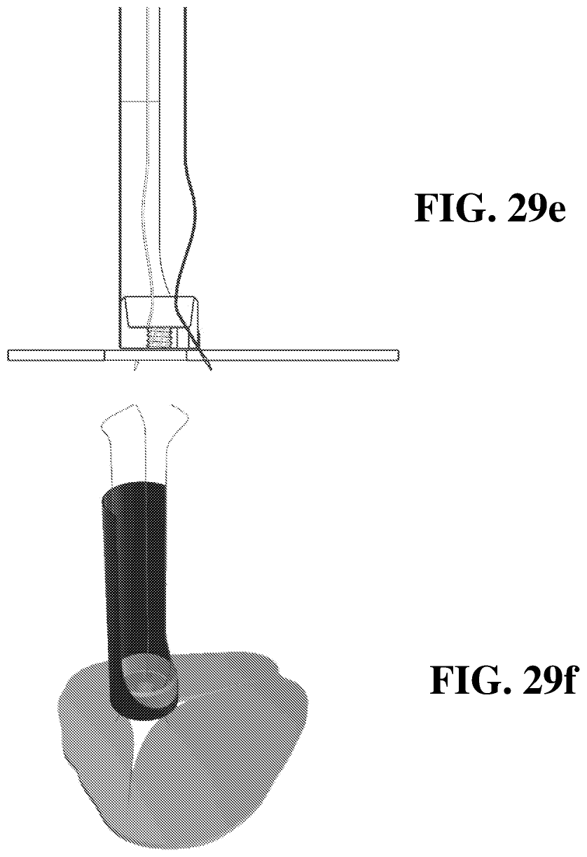

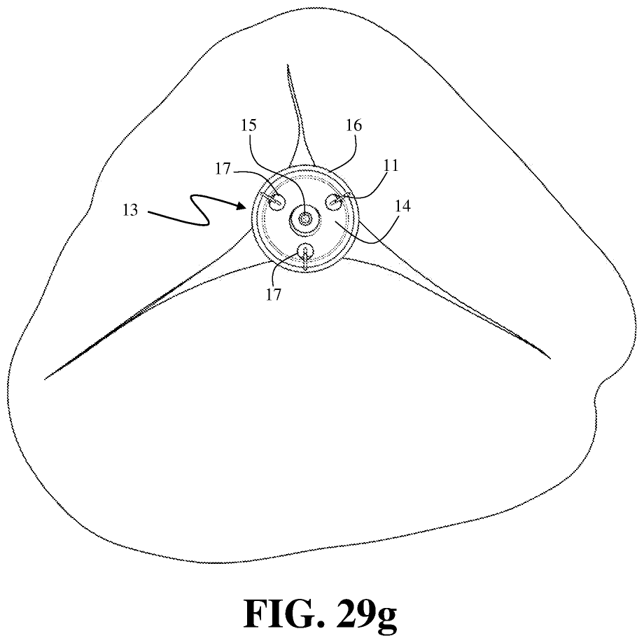

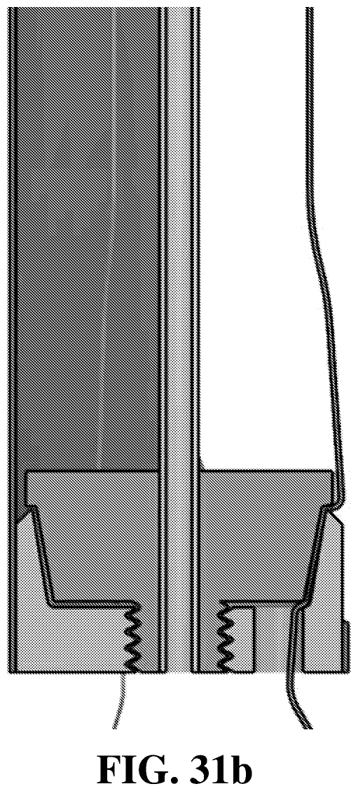

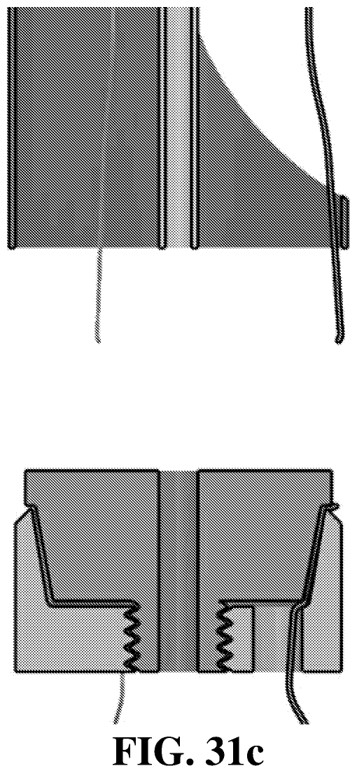

According to one aspect, the thread-like connection elements can be stretched and blocked by means of a holding device 12 and cut at a desired length by means of the same device 12 of the type illustrated in FIGS. 29a to 31c, or by means of another device.

According to one aspect, this holding device 12 includes a first element suitable for passing and containing the wires, a second element suitable for coupling with the first element so that by their union the thread-like connection elements are blocked and their sliding is prevented. In a further configuration, one of the two elements has a conformation of a portion that engages with the other element such that from their coupling it is also possible to obtain the cutting to size of the wires.

According to one merely illustrative aspect shown in the figures, the device 12 comprises at least one internally threaded countersunk washer 13, having a flat bottom plate 14 with a central axial through hole 15 with nut thread, which crosses it perpendicularly, and having a side wall 16 which rises from the flat bottom plate 14 along a peripheral area of the flat plate 14 itself. In the flat bottom plate 14 there is at least a second through hole 17, in an off-center position with respect to the axial central through hole 15, configured to be crossed by at least one suture thread 11. The countersunk washer 13 is configured to be inserted into the external catheter, which optionally can be the same external catheter 2 of the device 1, keeping the central through hole 15 coaxial to the catheter itself. As shown in FIGS. 29a and 29b, from the outside of the patient's body the countersunk washer 13 is inserted into the catheter with at least one suture thread 11 running in the respective second through hole 17.

From the outside of the patient's body (FIGS. 29a to 29g) the thread-like connection elements 11 are inserted into the holes 17 of the countersunk washer 13 and the countersunk washer 13 is guided to the center of the valve to be repaired (FIG. 29g). Preferably, on the flat plate 14 at the bottom of the countersunk washer 13 there are as many second through holes 17 as the thread-like connection elements 11 to be stretched and cut, so that each thread-like connection element is inserted into the respective second through hole 17. In the example shown in FIG. 29g, the flat bottom plate 14 of the countersunk washer 13 has holes for the suture threads 11 arranged with angular symmetry with respect to the central through hole 15 of the countersunk washer 13. An advantage of the symmetrical arrangement consists in the fact that, by stretching the thread-like connection elements at the same time, the countersunk washer 13 automatically positions itself so that its central through hole 15 is in the middle of the thread-like connection elements 11. However, it is possible to make only one hole 17 (distinct from the central through hole 15) to pass all the thread-like connection elements 11 through it, but in this case the position of the central through bole 15 will not be centered between the thread-like connection elements 11.

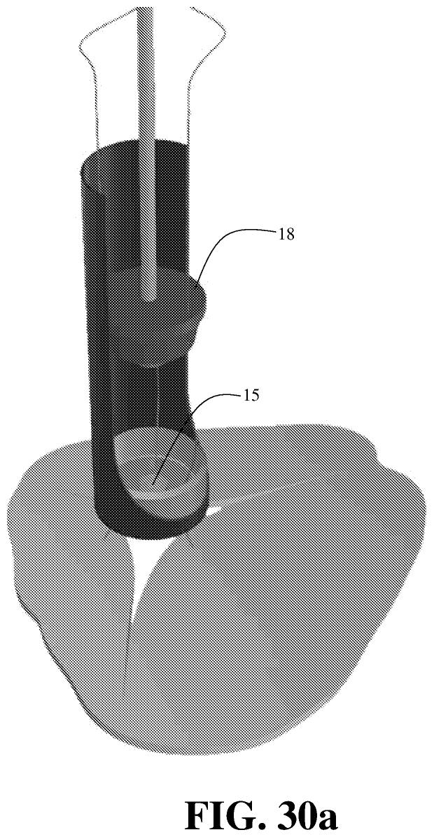

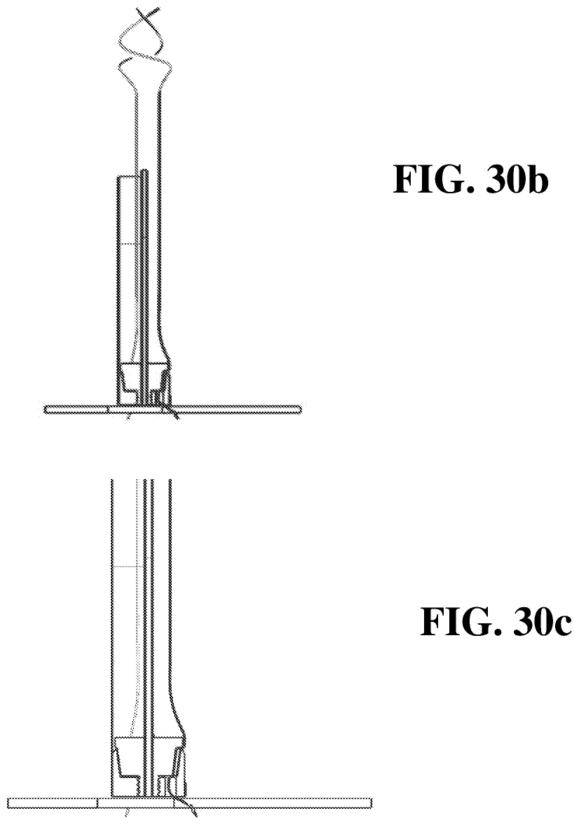

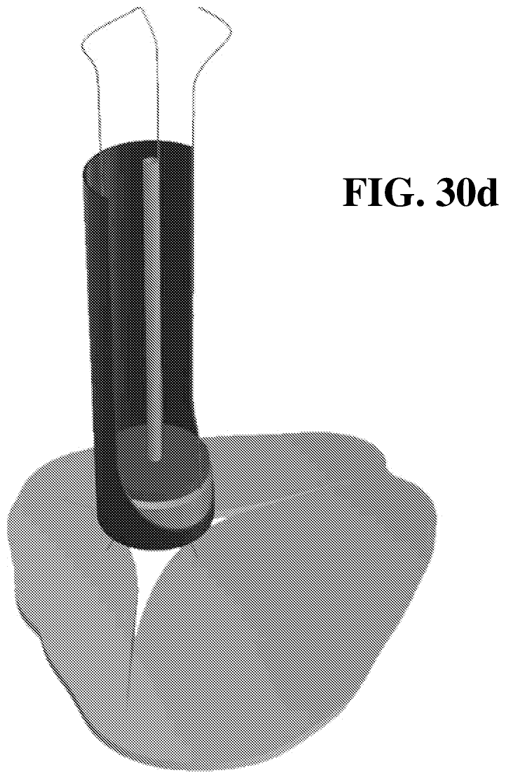

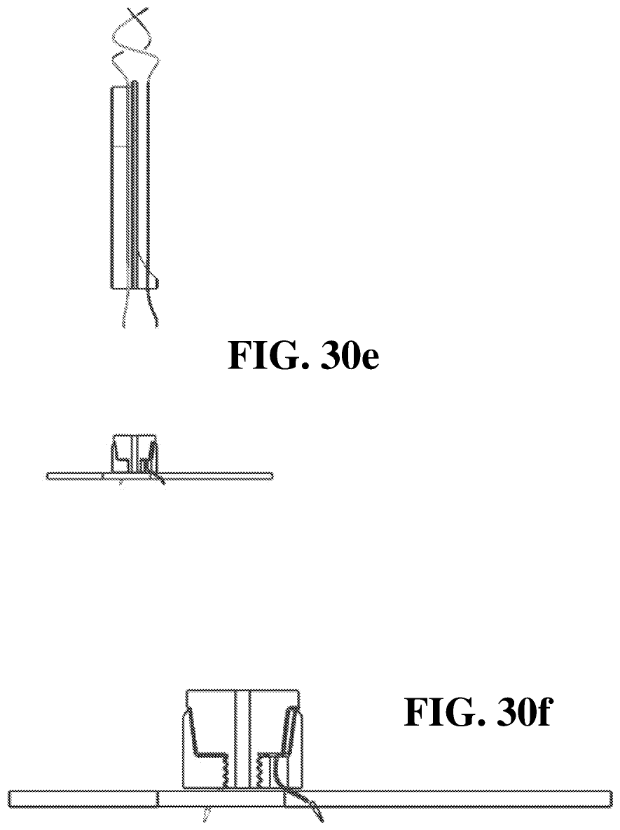

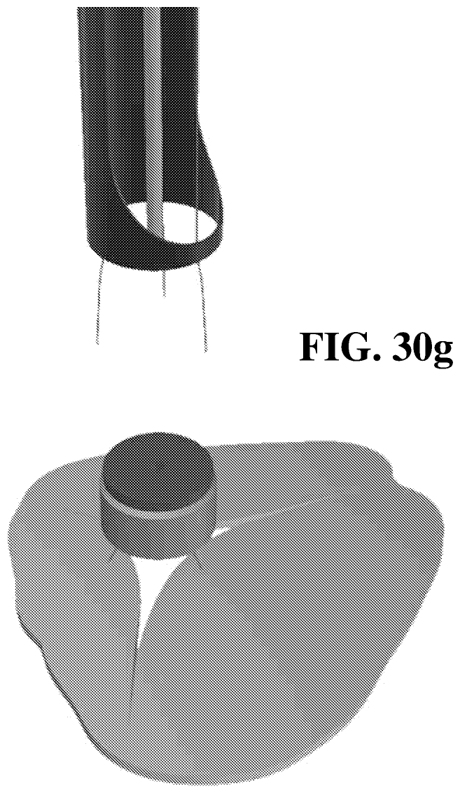

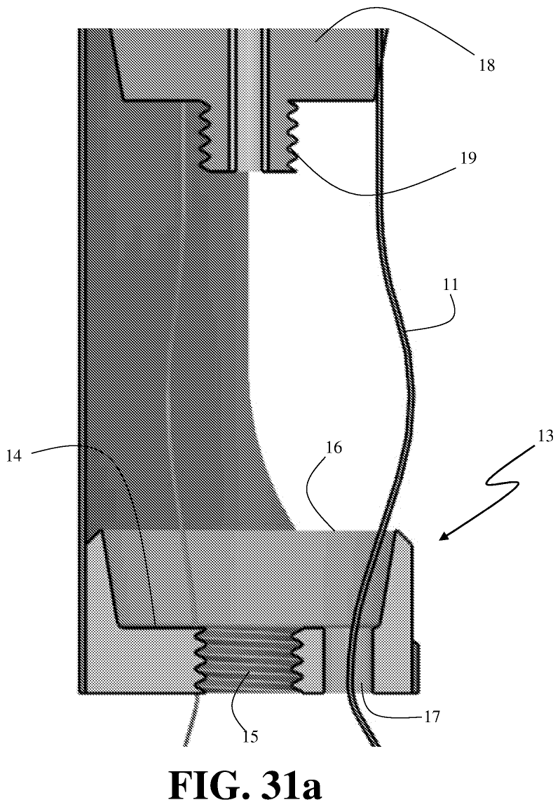

Once the countersunk washer 13 is substantially positioned in the valve plane, the thread-like connection elements are pulled axially with respect to the catheter so that the flaps are extended in the valve plane and a screw is coaxially advanced into the catheter (FIGS. 30a to 30d) having a stem 19 which can be screwed into the central through hole 15 and having a head 18 configured to rest on a free edge of the side wall 16 of the countersunk washer 13. By tightening the screw in the internally threaded countersunk washer 13, the head of the screw 18 is pressed against the free edge of the countersunk washer 13 and together cooperate to cut the thread-like connection elements 11, which remain trapped between them (FIGS. 30e to 30g) keeping the flaps extended.

Optionally, the free edge of the side wall 16 of the countersunk washer 13 will have a profile shaped like a blade so as to facilitate the cutting of the thread-like elements 11 when the screw is tightened, as can be seen in the detail views of the figures from 31a to 31c. Once the thread-like connection elements 11 have been cut, the external catheter and the suture threads are withdrawn, leaving the countersunk washer 13 and the screw tightly screwed therein in the valve plane.

The device 1 for joining the flaps of a tricuspid or mitral valve can optionally be distributed as part of a kit for surgical operations, comprising the device 1 itself and a device 12 for cutting and holding at least one thread-like connection element, comprising:
an external catheter 2 adapted to be inserted into a patient's blood vessel;
an internally threaded countersunk washer 13, having a flat bottom plate 14 with a central axial through hole 15 with a nut thread which crosses substantially perpendicularly the flat plate 14, and having a side wall 16 which rises from the flat bottom plate 14 along a peripheral region of the flat bottom plate 14, the flat bottom plate 14 having at least a second through hole 17 configured to be crossed by at least one thread-like connection element 11, the countersunk washer 13 being configured to be inserted into the external catheter 2 while maintaining the central through hole 15 coaxially with the external catheter 2;
a screw configured to coaxially advance into the external catheter, having a shaft 19 screwable into the central through hole 15 and having a head 18 configured to rest on a free edge of the side wall 16 of the countersunk washer 13 and to cooperate with the free edge of the countersunk washer 13 to cut the thread-like element held between the countersunk washer 13 and the head 18 of the screw.

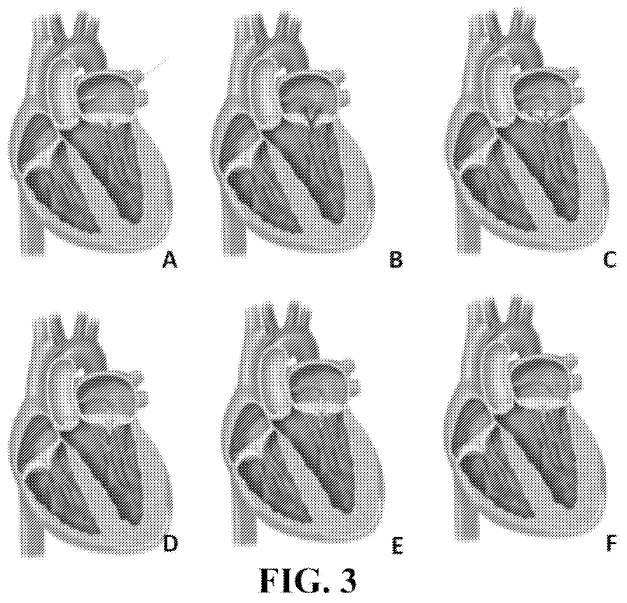

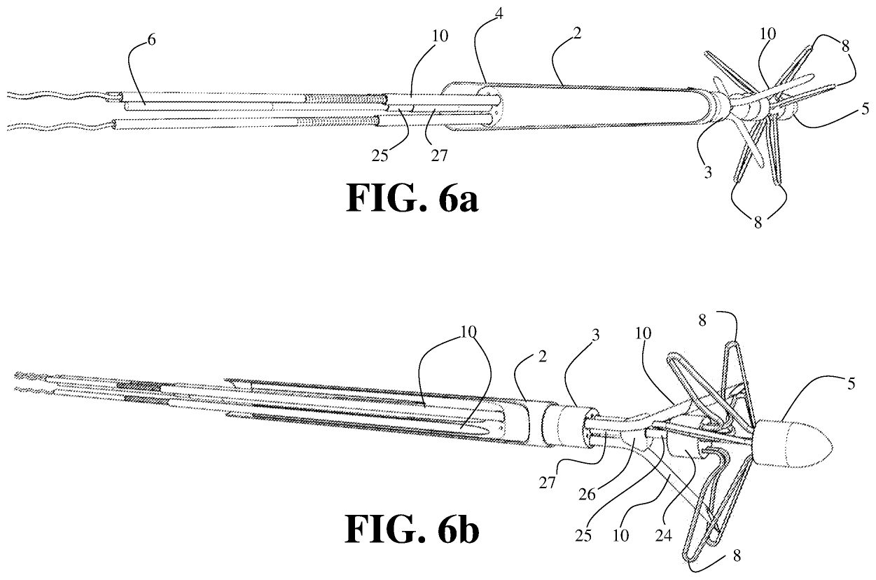

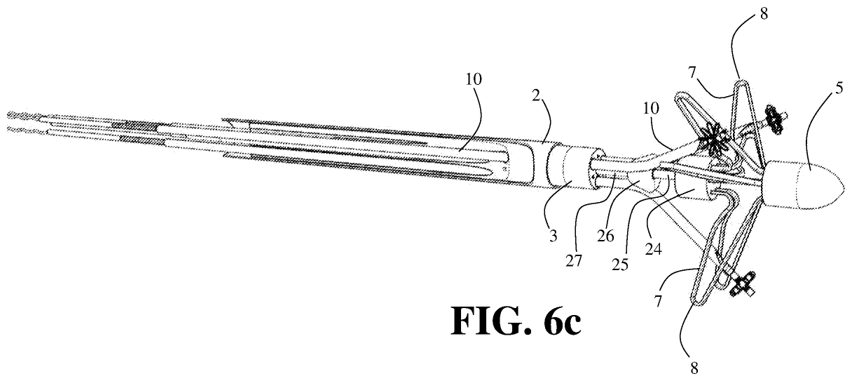

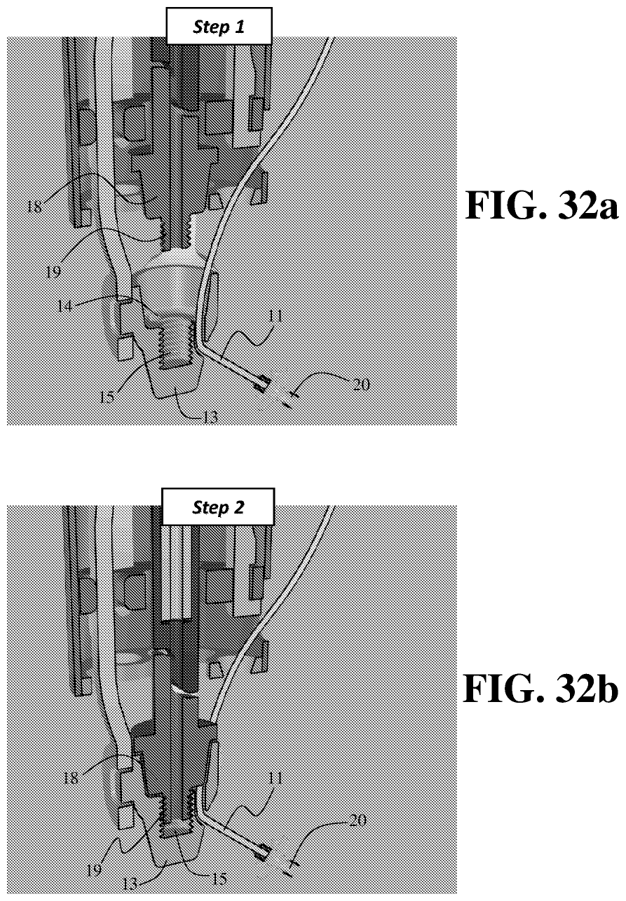

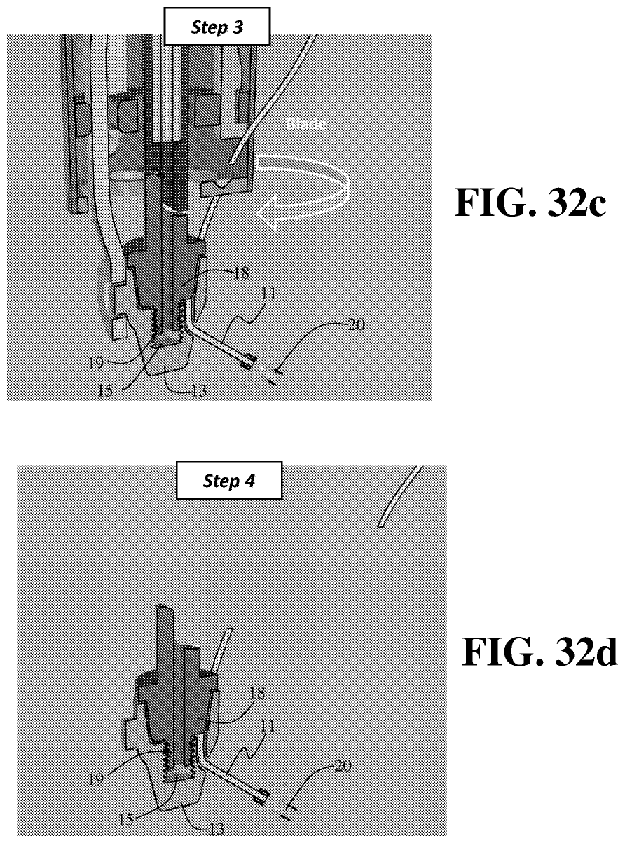

According to one aspect, a holding device according to another form of the present disclosure can be made in the manner illustrated in FIGS. 32a to 32d. The operation of this other embodiment is similar to that of the cutting and holding device shown in FIGS. 28a to 31c and can be used with the device of FIG. 6a. The device of FIGS. 32a to 32d is also based on the cooperation between a screw and a countersunk washer 13 with a central hole 15, which in the figures is a blind hole 15, with a nut screw thread. Unlike the device of FIGS. 3la to 31c, in the embodiment illustrated in FIGS. 32a to 32d the countersunk washer 13 with a central hole 15 and the screw inserted therein serve only to hold the thread-like connection elements and not to cut them. The thread-like connection elements are cut because the illustrated cutting and holding device has a blade (FIG. 32c) which can rotate circumferentially to sever the thread-like connection elements protruding from the countersunk washer (FIG. 22d).

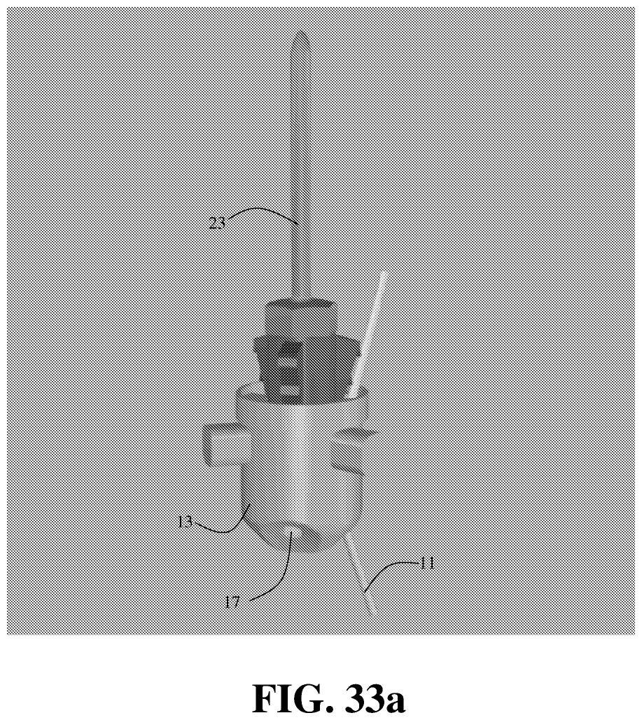

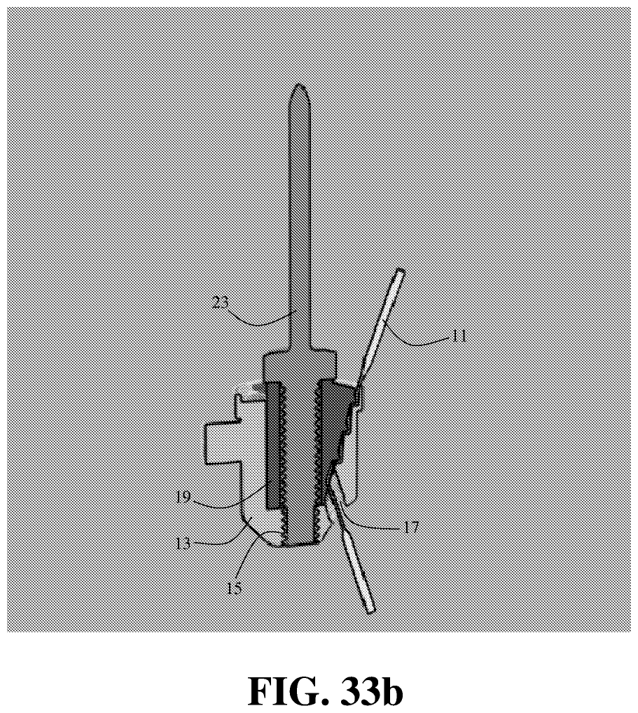

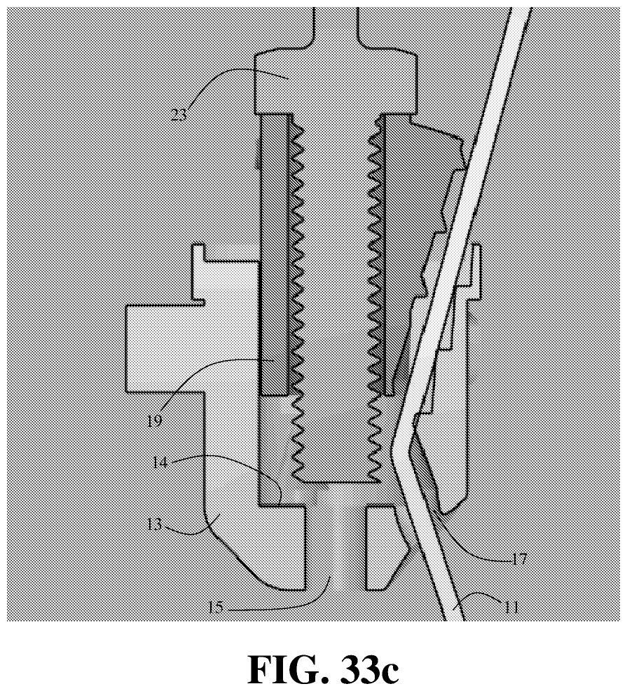

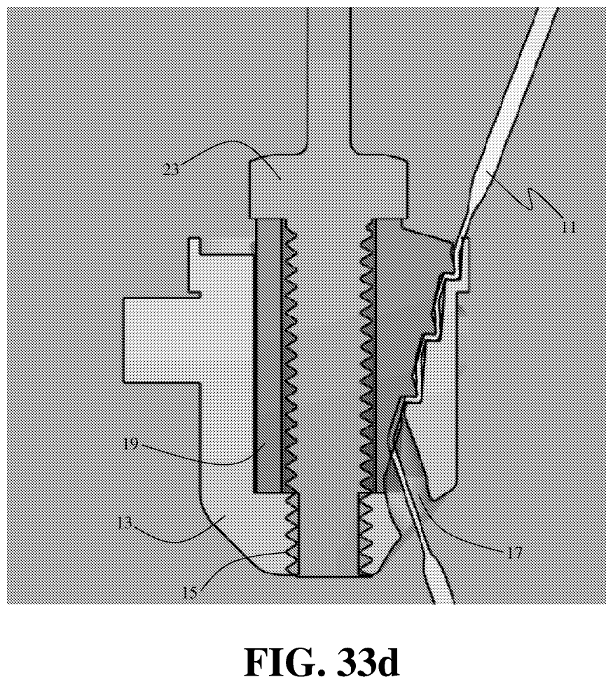

According to yet another embodiment of the cutting and holding device illustrated in FIGS. 33a to 33d, the stem 19 of the screw inserted in the central through hole 15 of the washer 13 is hollow and internally threaded with a nut screw. Thanks to a threaded pin 23, the screw can be simply inserted into the hole of the washer and firmly joined to the washer 13 by screwing the threaded pin 23 into the nut screw hole of the washer 13. Once this operation is finished, the thread-like connection elements are cut with a blade which can be rotated circumferentially as in the device of FIG. 32c and the threaded pin 23 is left in place (FIG. 33b).

According to one aspect shown in the sectional views of FIGS. 33c and 33d, the countersunk washer 13 has an internal stepped profile mating with an external stepped profile of the screw, so as to crush and hold the thread-like connection elements in several points, preventing them accidentally from slipping off.

According to one aspect, the holding device 12 of at least one thread-like connecting element 11 could be distributed separate from the device for joining 1 of this disclosure and used in other devices for tensioning and cutting wires during surgical operations. Any variations or additions can be made by experts in the technical field to the embodiments described and illustrated here, while remaining within the scope of the following claims. In particular, further embodiments may include the technical characteristics of one of the following claims with the addition of one or more technical characteristics described in the text or illustrated in the figures, taken individually or in any reciprocal combination.

1. A device for joining flaps of a heart valve, comprising:
an external catheter adapted to be inserted into a blood vessel of a patient;
a proximal body, defining a first central axial through hole which runs in the proximal body from a first base surface thereof to a second base surface thereof;
a deflector body defining a second central axial through hole which runs therethrough, said deflector body being configured so that, when the deflector body is outside said external catheter, an internal catheter pushed in said longitudinal direction slides in contact along a side surface of said deflector body and is forced to bend in a guided manner because of the contact against said side surface so as to diverge from a central longitudinal axis of the device;
an intermediate body, defining a third central axial through hole which runs in the intermediate body from a first base surface thereof to a second base surface thereof;
a distal body, having a coaxial rod which is fixed and rises from a central position of a base surface of the distal body, wherein said coaxial rod is slidably threaded into said third axial through hole of the intermediate body;
a plurality of arms, each arm of the plurality of arms having a first end fixed on the second base surface of the intermediate body and a second end fixed to said distal body, wherein each arm is configured to assume a radially expanded position when outside said external catheter, and to assume an extended position parallel to said coaxial rod when both the intermediate body and the distal body are inside said external catheter, wherein said arms are configured so that, when in said radially expanded position, they define radially oriented supports for supporting flaps of a heart valve;
wherein said proximal body defines a plurality of longitudinal through channels which run in the proximal body from as many inlet holes in the first base surface of the proximal body, to as many outlet holes in the second base surface of the proximal body, wherein said through channels are oriented along said longitudinal direction from said inlet holes to said outlet holes;
a plurality of internal catheters, each internal catheter of said internal catheters is connected to a respective inlet hole of said inlet holes and is configured to convey a respective filiform connection element or a respective inflation tube to a respective through channel of said through channels and to cause said respective filiform connection element or said respective inflation tube to come out of the respective outlet hole from the second base surface of the proximal body, said internal catheters being configured to bend, because of the contact against the side surface of said deflector body, so as to diverge from a central longitudinal axis of the device when pushed in said longitudinal direction to slide in contact along the side surface of said deflector body.

2. The device according to claim 1, wherein said deflector body has a side surface which defines a minor cross section at a proximal portion of the deflector body and a major cross section at a distal portion of the deflector body.

3. The device according to claim 2, wherein said deflector body is an element that can be inflated/deflated through a first coaxial tube slidably inserted into said first central axial through hole of the proximal body, in which said inflatable/deflatable member when inflated surrounds said second coaxial tube and has said side surface defining a minor cross section at a proximal portion of the inflated member and a major cross section at another portion of the inflated member.

4. The device according to 1, wherein:
said intermediate body has a second coaxial tube which is fixed and rises from a central position of the first base surface of the intermediate body and is in communication with said third axial through hole and is slidably inserted in said second central axial through hole; and
said coaxial rod is slidably inserted into said second coaxial tube.

5. The device (1) according to claim 1, wherein said intermediate body is fixed to said proximal body and is kept at a predetermined distance from said proximal body through a plurality of bars arranged in the longitudinal direction and configured to engage the side surface of said deflector body preventing said deflector body from rotating about the longitudinal direction.

6. The device according to claim 1, wherein:
said deflector body is a movable solid and has a transverse minor base surface, a transverse major base surface and said side surface of the deflector body between the transverse minor base surface and the transverse major base surface,
said movable solid has a first coaxial tube which is fixed and rises from a central position of the minor base surface and is in communication with said second axial through hole, and
said first coaxial tube is slidably inserted into said first central axial through hole of the proximal body.

7. The device according to claim 1, wherein said deflector body comprises:
a frame fixed to transversely surround said proximal body,
a plurality of fins and as many rings, each fin of said fins having a first end fixed to said frame and a second end, opposite to said first end, integral with a respective ring of said rings;
wherein an internal catheter of said plurality of internal catheters is inserted into a respective ring of said rings;
wherein each ring of said rings is configured so that, when the respective internal catheter is pushed in said longitudinal direction, it is guided to slide along the respective fin of said fins of the deflector body;
wherein said plurality of fins is made of shape memory material and is configured in such a way that, as the fins come out of the external catheter, the respective second ends move further and further away so as to diverge from a central longitudinal axis of the device by mutually distancing said rings; and
wherein said second coaxial tube is fixed to said proximal body so as to keep said intermediate body integral with and at a fixed distance from said proximal body.

8. The device according to claim 1, further comprising a plurality of thread-like connection elements or a plurality of inflation tubes, wherein each thread-like connection element of said thread-like connection elements or each inflation tube of said inflation tubes is inserted into a respective internal catheter of said internal catheters, said filiform connection elements or inflation tubes having terminations with hooking elements configured, when exiting from the respective internal catheters, to engage the flaps of said heart valve and to expand autonomously in the radial direction after having hooked said flaps so that, by pulling the filiform connection elements or the inflation tubes, the flaps of the valve are dragged by said hooking elements approaching each other towards the central longitudinal axis of the device.

9. The device according to claim 1, wherein each arm of the plurality of arms is integral and consists of:
a shape memory material; or
a deformable material configured to elastically return to a resting shape.

10. The device according to claim 9, wherein each arm of the plurality of arms is configured so that, by progressively approaching the intermediate body to the distal body, the arm passes from said extended position to said radially expanded position with an elbow bend and further to a closed position in which said elbow bend is functionally configured to cooperate with said coaxial rod to squeeze like two jaws a flap of a heart valve.

11. The device according to claim 1, wherein each arm of the plurality of arms consists of a first rigid rod hinged at said first end of the arm on the second base surface of the intermediate body, and a second rigid rod hinged at said second end of the arm to said distal body, said first rod and said second rod being rigid and mutually hinged at an intermediate zone between said first end and said second end.

12. The device according to claim 11, wherein said second rod has a hook-shaped free end, each arm being configured so that, by progressively approaching the intermediate body to the distal body, the arm passes from said extended position to said radially expanded position with an elbow bend and further to a closed position in which said hook-shaped free end is functionally configured to cooperate with said coaxial rod to squeeze like two jaws a flap of a heart valve.

13. The device according to claim 1, wherein each hooking element of said hooking elements is composed of a tubular body partially cut into longitudinal strips, wherein said longitudinal strips are made of elastic or shape memory material and are configured to lie longitudinally flat when in the respective internal catheter and to self-expand radially when out of the respective internal catheter.

14. The device according to claim 1, wherein each hooking element of said hooking elements is a balloon inflated through a respective inflation tube, said inflatable balloon being configured to remain deflated when in the respective internal catheter and to expand in a radial direction when, once out of the respective internal catheter, it is inflated.

15. The device according to claim 13, wherein said internal catheters have terminations shaped like a tip of a syringe needle and are configured to pierce through a flap of a heart valve.

16. The device according to claim 1, wherein said plurality of through channels comprises three through channels and said plurality of internal catheters comprises three internal catheters each of which is connected to an inlet hole of the respective through channel.

17. The device according to claim 1, further comprising cutting devices configured to cut said thread-like connection elements.

18. A kit for surgical operations, comprising a device for joining flaps of a heart valve according to claim 1 and a holding device for holding a one filiform connection element, in which said holding device comprises:
an internally threaded flared washer, having a flat bottom plate with a central axial hole with lead screw thread perpendicularly passing through said flat plate, and having a side wall which rises from said flat bottom plate along a peripheral zone of the flat bottom plate, said flat bottom plate having at least one second through hole configured to be crossed by at least one filiform connection element, said flared washer being configured to be inserted into said external catheter keeping said central through hole coaxial to the external catheter; and
a screw configured to advance coaxially in said external catheter and to be constrained to the flared washer so as to hold said filiform connection element squeezed between an inner surface of the side wall of the flared washer and a side surface of said screw.
